SEQUENCE LISTING (21 sequences)

SEQ ID NO: 1 - protein, 12 aa, ARTIFICIAL SEQUENCE
  synthetic polypeptide
ANIKLSVQMKLF

SEQ ID NO: 2 - protein, 12 aa, ARTIFICIAL SEQUENCE
  synthetic polypeptide
KLSVQMKLFKRH

SEQ ID NO: 3 - protein, 12 aa, ARTIFICIAL SEQUENCE
  synthetic polypeptide
VQMKLFKRHLKW

SEQ ID NO: 4 - protein, 12 aa, ARTIFICIAL SEQUENCE
  synthetic polypeptide
KLFKRHLKWKII

SEQ ID NO: 5 - protein, 12 aa, ARTIFICIAL SEQUENCE
  synthetic polypeptide
KRHLKWKIIVKL

SEQ ID NO: 6 - protein, 12 aa, ARTIFICIAL SEQUENCE
  synthetic polypeptide
LKWKIIVKLNDG

SEQ ID NO: 7 - protein, 12 aa, ARTIFICIAL SEQUENCE
  synthetic polypeptide
KIIVKLNDGREL

SEQ ID NO: 8 - protein, 12 aa, ARTIFICIAL SEQUENCE
  synthetic polypeptide
VKLNDGRELSLD

SEQ ID NO: 9 - protein, 12 aa, ARTIFICIAL SEQUENCE
  synthetic polypeptide
QMKLFKRHLKWK

SEQ ID NO: 10 - protein, 12 aa, ARTIFICIAL SEQUENCE
  synthetic polypeptide
MKLFKRHLKWKI

SEQ ID NO: 11 - protein, 14 aa, ARTIFICIAL SEQUENCE
  synthetic polypeptide
MKLFKRHLKWKIIV

SEQ ID NO: 12 - protein, 12 aa, ARTIFICIAL SEQUENCE
  synthetic polypeptide
XLFKRHLKWKII

SEQ ID NO: 13 - protein, 12 aa, ARTIFICIAL SEQUENCE
  synthetic polypeptide
KLFXRHLKWKII

SEQ ID NO: 14 - protein, 12 aa, ARTIFICIAL SEQUENCE
  synthetic polypeptide
KLFKRHLXWKII

SEQ ID NO: 15 - protein, 12 aa, ARTIFICIAL SEQUENCE
  synthetic polypeptide
KLFKRHLKWXII

SEQ ID NO: 16 - protein, 12 aa, ARTIFICIAL SEQUENCE
  synthetic polypeptide
KLFKKHLKWKII

SEQ ID NO: 17 - protein, 11 aa, ARTIFICIAL SEQUENCE
  synthetic polypeptide
KLFKHLKWKII

SEQ ID NO: 18 - protein, 4 aa, ARTIFICIAL SEQUENCE
  synthetic peptide substrate
IEAR

SEQ ID NO: 19 - protein, 33 aa, ARTIFICIAL SEQUENCE
  Bpep-25 peptide
ANIKLSVQMKLFKRHLKWKIIVKLNDGRELSLD

SEQ ID NO: 20 - protein, 18 aa, ARTIFICIAL SEQUENCE
  antimicrobial peptide
YGAAKKAAKAAKKAAKAA

SEQ ID NO: 21 - protein, 18 aa, ARTIFICIAL SEQUENCE
  antimicrobial peptide
YGAKAKAAKAAKAKAAKA

(19) United States
(12) Patent Application Publication
(10) Pub. No.: US 2005/0118678 A1
(43) Pub. Date: Jun. 2, 2005

(54) Modified polypeptides with therapeutic activity and methods of use

(75) Inventor: Kevin H. Mayo, Minneapolis, MN (US)

Correspondence Address: MUETING, RAASCH & GEBHARDT, P.A., P.O. BOX 581415, MINNEAPOLIS, MN, 55458, US

(73) Assignee: Regents of the University of Minnesota, Minneapolis, MN (US)

(21) Appl. No.: 10/967,060

(22) Filed: Oct. 15, 2004

Related U.S. Application Data
(60) Provisional application No. 60/512,372, filed on Oct. 17, 2003.

(51) Int. Cl. A61K 38/17; C07H 21/04; C07K 14/47

(52) U.S. Cl. 435/69.1; 435/320.1; 435/325; 514/12; 530/350; 536/23.5

(57) ABSTRACT

Polypeptides modified by fatty acid conjugation and methods of using such modified polypeptides in treating bacterial infections, including the treatment of antibiotic resistant bacterial infections, are disclosed.

### CONTINUING APPLICATION DATA

This application claims the benefit of U.S. Provisional Application Ser. No. 60/512,372, filed Oct. 17, 2003, which is incorporated by reference herein.

### BACKGROUND

The triumph of antibiotics over disease-causing bacteria is one of modern medicine's greatest success stories. Since these drugs first became widely used in the World War II era, they have saved countless lives and blunted serious complications of many feared diseases and infections. However, after more than fifty years of widespread use many antibiotics are losing effectiveness. Disease-causing microbes have become resistant to drug therapy and are an increasing public health problem. Diseases such as tuberculosis, gonorrhea, malaria, and childhood ear infections are now more difficult to treat than they were decades ago. Drug resistance (also known as antibiotic resistance or antimicrobial resistance) is an especially difficult problem for hospitals, as hospitals harbor the critically ill patients who are more vulnerable to infections than the general population and therefore require more antibiotics. Heavy use of antibiotics in these patients hastens the mutations in bacteria that bring about drug resistance. Unfortunately, this worsens the problem by producing bacteria with greater ability to survive treatment with the strongest antibiotics. These even stronger drug-resistant bacteria continue to prey on vulnerable hospital patients.

Organisms that have developed defenses against antibiotics include Staphylococcus aureus, Enterococcus, Streptococcus pneumoniae (which can cause pneumonia, meningitis, and ear infections), Neisseria gonorrhoeae (cause of the sexually transmitted disease gonorrhea), Salmonella, Escherichia coli (E. coli), and Mycobacterium tuberculosis (which causes tuberculosis).

According to the Centers for Disease Control and Prevention (CDC), nearly two million patients in the United States get an infection in the hospital each year. Of those patients, about 90,000 die each year as a result of their infection, up from 13,300 patient deaths in 1992. More than 70 percent of the bacteria that cause hospital-acquired infections are resistant to at least one of the drugs most commonly used to treat them. Persons infected with drug-resistant organisms are more likely to have longer hospital stays and require treatment with second or third choice drugs that may be less effective, more toxic, and more expensive. In short, antimicrobial resistance is driving up health care costs, increasing the severity of disease, and increasing the death rates from certain infections.

There are several signs that the problem of bacterial resistance to antibiotics is increasing. In 2003, epidemiologists reported in The New England Journal of Medicine that five to ten percent of patients admitted to hospitals acquire an infection during their stay, and that the risk for a hospital-acquired infection has risen steadily in recent decades. Strains of S. aureus resistant to methicillin are endemic in hospitals and are increasing in non-hospital settings such as locker rooms. Since September 2000, outbreaks of methicillin-resistant S. aureus infections have been reported among high school football players and wrestlers in California, Indiana, and Pennsylvania, according to the CDC. The first S. aureus infections resistant to vancomycin emerged in the United States in 2002, presenting physicians and patients with a serious problem. Increasing reliance on vancomycin has led to the emergence of vancomycin-resistant enterococci infections. Prior to 1989, no U.S. hospital had reported any vancomycin resistant enterococci, but over the next decade, such microbes become common in U.S. hospitals, according to CDC.

Resistance to the antibiotics currently being used to treat human illnesses is a serious public health threat. Thus, there is a need for new and improved antibacterial agents.

### SUMMARY OF THE INVENTION

The present invention provides a modified polypeptide having a polypeptide having an amphipathic α-helical or 3₁₀ helical structure having one surface comprising primarily positively charged amino acid residues and an opposing surface comprising primarily hydrophobic amino acid residues, wherein these residues define a surface active domain, wherein the polypeptide has up to 14 amino acid residues, wherein the polypeptide has been modified at the N-terminus and/or the C-terminus to include a linear or branched aliphatic group having at least 6 carbon atoms, and wherein the modified polypeptide demonstrates enhanced bactericidal activity compared to the bactericidal activity of the polypeptide prior to modification at the N-terminus and/or the C-terminus to include a linear or branched aliphatic group having at least 6 carbon atoms. In some embodiments of the modified polypeptide, the polypeptide is selected from SEQ ID NOs: 1-17 or an active analog thereof, wherein X is an amino acid, and wherein an active analog thereof includes the deletion of one or two contiguous or noncontiguous amino acid residues, the addition of one or two contiguous or noncontiguous amino acids, the substitution of one or two amino acids, chemical modification, and/or enzymatic modification. In some embodiments, X may be norleucine. In some embodiments, the polypeptide may be selected from SEQ ID NOs: 1-8 or an active analog thereof, wherein an active analog thereof includes the deletion of one or two contiguous or noncontiguous amino acid residues, the addition of one or two contiguous or noncontiguous amino acids, the substitution of one or two amino acids, chemical modification, and/or enzymatic modification.

The present invention also provides a modified polypeptide having a polypeptide having a beta sheet structure having one surface comprising primarily positively charged amino acid residues and an opposing surface comprising primarily hydrophobic amino acid residues, wherein these residues define a surface active domain, wherein the polypeptide has up to 14 amino acid residues, wherein the polypeptide has been modified at the N-terminus and/or the C-terminus to include a linear or branched aliphatic group having at least 6 carbon atoms, and wherein the modified polypeptide demonstrates enhanced bactericidal activity compared to the bactericidal activity of the polypeptide prior to modification at the N-terminus and/or the C-terminus to include a linear or branched aliphatic group having at least 6 carbon atoms.

The present invention also provides a modified polypeptide having a polypeptide with a sequence selected from SEQ ID NOs: 1-17 or an active analog thereof, wherein X is an amino acid, wherein the polypeptide has been modified at the N-terminus and/or the C-terminus to include a linear or branched aliphatic group having at least 6 carbon atoms, wherein an active analog includes the deletion of one or two contiguous or noncontiguous amino acid residues, the addition of one or two contiguous or noncontiguous amino acids, the substitution of one or two amino acids, chemical modification, and/or enzymatic modification. In some embodiments, X is norleucine. In some embodiments, the polypeptide is selected from SEQ ID NOs: 1-8 or an active analog thereof. In some embodiments, the modified polypeptide has SEQ ID NO:4 or an active analog thereof. In some embodiments, the modified polypeptide is SEQ ID NO:4.

In some embodiments of the modified polypeptides of the present invention, the modified polypeptide demonstrates enhanced bactericidal activity compared to the bactericidal activity of the polypeptide prior to modification at the N-terminus and/or the C-terminus to include a linear or branched aliphatic group having at least 6 carbon atoms.

In some embodiments of the modified polypeptides of the present invention, the modified polypeptide may be about 8 to about 33 amino acids in length, may be about 10 to about 25 amino acids in length, or may be about 12 to about 14 amino acids in length. In some embodiments, a modified polypeptide of the present invention may have up to about 14 amino acid residues, up to about 12 amino acid residues, or up to about 10 amino acid residues. In some embodiments, a modified polypeptide is a dodecamer, having twelve amino acids residues.

In some embodiments of the modified polypeptides of the present invention, the aliphatic group includes one or more unsaturated carbon-carbon bonds. In some embodiments, the aliphatic group may have at least 11 carbon atoms. In some embodiments, the aliphatic group may have about 11 to about 19 carbon atoms. In some embodiments, the aliphatic group may be bonded to the polypeptide at the N-terminus or C-terminus. In some embodiments, the aliphatic group is an alkyl group derived from a fatty acid, including, for example, a C8-C22 fatty acid, a C10-C20 fatty acid, or a C8-C22 fatty acid.

The present invention includes a composition including one or more modified polypeptides. The present invention also includes a composition including one or more modified polypeptides and a pharmaceutically acceptable carrier.

The present invention includes a method for treating a bacterial infection in a subject by administering to a subject a modified polypeptide in an amount effective to demonstrate bactericidal activity. In some embodiments, the modified polypeptide may also neutralizes endotoxin.

The present invention includes a method for treating endotoxemia in a subject by administering to a subject a modified polypeptide in an amount effective to neutralize endotoxin. In some embodiments, the modified polypeptide may also demonstrate bactericidal activity.

The present invention includes a method for inhibiting bacterial growth in vitro by contacting bacteria with a modified polypeptide in an amount effective to inhibit bacterial cell growth and/or demonstrate bactericidal activity.

The present invention includes a method for neutralizing endotoxin in vitro by contacting cells with a modified polypeptide in an amount effective to neutralize endotoxin.

The present invention includes a method for decreasing the amount of TNFα in a subject by administering to the subject a modified polypeptide of claim 1 in an amount effective to decrease the amount of TNFα.

The present invention includes a method for decreasing the amount of TNFα in vitro by incubating cells with a modified polypeptide in an amount effective to decrease the amount of TNFα.

The present invention includes a method for inhibiting endothelial cell proliferation in a subject by administering to the subject a modified polypeptide in an amount effective to inhibit endothelial cell proliferation.

The present invention includes a method for inhibiting endothelial cell proliferation in vitro by contracting endothelial cells with a modified polypeptide in an amount effective to inhibit endothelial cell proliferation.

The present invention includes a method for inhibiting angiogenic-factor mediated inter-cellular adhesion molecule expression down-regulation in a subject by administering to the subject a modified polypeptide in an amount effective to inhibit angiogenic-factor mediated inter-cellular adhesion molecule expression down-regulation.

The present invention includes a method for inhibiting angiogenic-factor mediated inter-cellular adhesion molecule expression down-regulation in vitro by contacting endothelial cells with a modified polypeptide in an amount effective to inhibit angiogenic-factor mediated inter-cellular adhesion molecule expression down-regulation.

The present invention includes a method for inhibiting angiogenesis in a subject by administering to the subject a modified polypeptide in an amount effective to inhibit angiogenesis.

The present invention includes a method for inhibiting angiogenesis in vitro by contacting cells with a modified polypeptide in an amount effective to inhibit angiogenesis.

The present invention includes a method for inhibiting tumorigenesis in a subject by administering to the subject a modified polypeptide in an amount effective to inhibit tumorigenesis.

As used herein, “a” or “an” refers to one or more of the term modified. Thus, the compositions of the present invention include one or more modified polypeptides.

“Amino acid” is used herein to refer to a chemical compound with the general formula: NH₂—CRH—COOH, where R, the side chain, is H or an organic group. Where R is an organic group, R can vary and is either polar or nonpolar (i.e., hydrophobic). The amino acids of this invention can be naturally occurring or synthetic (often referred to as nonproteinogenic). As used herein, an organic group is a hydrocarbon group that is classified as an aliphatic group, a cyclic group or combination of aliphatic and cyclic groups. The term “aliphatic group” means a saturated or unsaturated linear or branched hydrocarbon group. This term is used to encompass alkyl, alkenyl, and alkynyl groups, for example. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” refers to mono- or polycyclic aromatic hydrocarbon groups. As used herein, an organic group can be substituted or unsubstituted.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring.

The following abbreviations are used throughout the application:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE FIGURES

FIG. 1. Schematic of the peptide sequence (SEQ ID NO:4), peptide-amphiphile structures, and nomenclature. The “—NH₂” at the right of each sequence indicates amidation of the C-terminus.

FIG. 2. Representative dose-response curves of SC4 (●), C12-SC4 (▪), and C18-SC4 (▴) against gram-negative E. coli J96, gram-positive S. pyogenes Eaton, and drug resistant, gram positive S. aureus W73 134 bacteria. Lines are sigmoidal curve fits used to determine LD₅₀ values.

FIG. 3. CD spectra of SC4 (solid lines), C12-SC4 (dashed lines), and C18-SC4 (dotted lines) in DPC micelles, SDS micelles, DPPC liposomes, and DPPE/DPPG liposomes. In micellar membrane mimics, the SC4 amphiphiles showed spectra consistent with a helical conformation in DPC micelles and somewhat less helical conformation in SDS micelles. Liposome membrane mimics showed spectra indicating little SC4 amphiphile structure in DPPC liposomes (the red blood cell mimic), but a more structured state in bacterial-mimicking DPPE/DPPG liposomes. The SC4 peptide spectra indicate little structure under any condition. Spectra in water looked similar to those in DPPC liposomes; results at 37° C. were similar for all conditions.

FIGS. 4A-4D. Regions of TOCSY and NOESY spectra for C12-SC4 in SDS and DPC micelles. FIG. 4A represents TOCSY spectra for C12-SC4 in SDS micelles. FIG. 4B represents TOCSY spectra for C12-SC4 in DPC micelles. FIG. 4C represents NOESY spectra for C12-SC4 in SDS micelles. FIG. 4D represents NOESY spectra for C12-SC4 in DPC micelles. The increased dispersion of the TOCSY spectrum and more long-range NOEs indicate a more structured state for C12-SC4 in DPC micelles.

FIG. 5. αH and NH chemical shifts for C12-SC4 in DPC or SDS relative to SC4 under the same conditions. The upfield nature of the shifts indicates stabilization of helical conformation, especially in residues K1-H7, as a result of fatty acid conjugation.

FIG. 6. NOE connectivity for C12-SC4 in DPC and SDS micelles. The number and regularity of NOE connectivities in DPC suggests an ordered state, and the patterns are consistent with an α-helical conformation in residues K1 through K8. In SDS, there were far fewer observable NOEs, although several of them are consistent with a somewhat structured peptide.

FIGS. 7A-7C. NOE-derived structures of C12-SC4 in DPC micelles. FIG. 7A represents the superposition of the 24 final structures, using residues K1 through W9 for alignment. FIG. 7B is a ribbon backbone representation of one structure, showing the overall helical fold and a less-ordered C-terminus. Polar residues are shown in black, apolar residues in grey. The fatty acid tail is shown as ball-and-sticks. FIG. 7C represents an axial view of the average structure, demonstrating the distribution of charged side-chains (arginine and lysine) in an amphipathic helix. Polar residues are shown in black, apolar residues in grey.

FIG. 8. Side-chain chemical shift differences between C12-SC4 in SDS and DPC micelles and SC4 peptide under the same conditions. All chemical shift differences are given as absolute values to avoid speculation into reasons for positive and negative values.

FIG. 9. The lysine side-chain amine region of TOCSY spectra for C12-SC4 in DPC and SDS micelles.

FIG. 10. Antibacterial activity of SC4 (▪), C12-SC4 (●), and C18-SC4 (▴) against Gram-positive MN8 bacteria.

FIG. 11. Antibacterial activity of SC4 (▪), C12-SC4 (●), and C18-SC4 (▴) against Gram-positive Hoch bacteria.

FIG. 12. Antibacterial activity of SC4 (▪), C12-SC4 (●), and C18-SC4 (▴) against Gram-positive Knutson bacteria.

FIG. 13. Antibacterial activity of SC4 (▪), C12-SC4 (●), and C18-SC4 (▴) against Gram-positive FRI 722 bacteria.

FIG. 14. Antibacterial activity of SC4 (▪), C12-SC4 (●), and C18-SC4 (▴) against Gram-positive RN6390 bacteria.

FIG. 15. Antibacterial activity of SC4 (▪), C12-SC4 (●), and C18-SC4 (▴) against Gram-positive, drug resistant W73134 bacteria.

FIG. 16. Antibacterial activity of SC4 (▪), C12-SC4 (●), and C18-SC4 (▴) against Gram-positive, drug resistant M49780 bacteria.

### DETAILED DESCRIPTION OF ILLUSTRATIVE EMBODIMENTS OF THE INVENTION

The present invention is directed to polypeptides that have been modified by fatty acid conjugation. Such polypeptides that have been modified by fatty acid conjugation are referred to herein as “modified polypeptides.” Such modified polypeptides are preferably “peptide-amphiphiles” and demonstrate enhanced bactericidal activity, especially against Gram-positive bacteria, and/or enhanced endotoxin neutralization. Modified polypeptides of the present invention may also inhibit endothelial cell proliferation, inhibit angiogenic-factor mediated inter-cellular adhesion molecule down-regulation, inhibit angiogenesis, and/or inhibit tumorgenesis. The modified polypeptides of the present invention may also demonstrate antifungal and/or antiparasitic activity.

The modified polypeptides of the present invention can be used in a variety of applications, which can be therapeutic, prophylactic, or diagnostic. As used herein, “treating” a condition or a subject includes therapeutic, prophylactic, and diagnostic treatments. For example, the modified polypeptides of the present invention may be particularly effective in the treatment of bacterial infections, including the treatment of infections with antibiotic resistant bacteria.

The modified polypeptides of the present invention may also be used as antibacterial agents in a variety of applications. For example, one or more modified polypeptides of the present invention may be added to a composition to act as an antibacterial additive. Or, one or more modified polypeptides may be coated onto a surface, for example, onto the surface of a medical device, to provide an antibacterial activity to the coated surface.

### Polypeptides

A modified polypeptide of the present invention includes as one aspect a polypeptide to which a fatty acid is conjugated. This polypeptide may have a structure as previously described in WO 01/53335, U.S. patent application Ser. No. 20020146406, Mayo et al., Biochem. J. 349, 717-728 (2000), and Lockwood et al., Biochem. J. 378, 93-103 (2004). Briefly, the polypeptide aspect of the modified polypeptides of the present invention may be a polypeptide having an amphipathic structure having one surface having primarily positively charged amino acid residues and an opposing surface having primarily hydrophobic amino acid residues, wherein these residues define a surface active domain.

WO 01/53335 provides detailed information about the shape and structure of a surface active domain. As used herein, the term “surface active domain” refers to a region of a molecule or molecular complex that, as a result of its shape, demonstrates antibacterial activity and/or is active for the treatment of one or more conditions, such as those described herein. Such a surface active domain is the part of the polypeptide that is believed to be important for imparting the desired function. Thus, the structural information of just this domain can be used for identifying candidate polypeptides. “Structure coordinates” refers to Cartesian coordinates derived from computational modeling using internuclear distances obtained from NMR spectroscopic experiments. The structure coordinates generate a unique configuration of points in space. It should be noted that these coordinates represent a statistical best fit representation of numerous structures for any one polypeptide, and that slight variations in individual structure coordinates would be expected. Also, similar or identical configurations can be defined by an entirely different set of coordinates, provided the distances and angles between coordinates remain essentially the same.

Generally, the structure is an amphipathic structure, such as a helix (which can be viewed as a cylinder) or a beta sheet, wherein one surface includes primarily positively charged amino acid residues (preferably, one surface is composed primarily of positively charged amino acid residues (i.e., hydrophilic amino acid residues)) and the opposing surface includes hydrophobic amino acid residues (preferably, the opposing surface is composed primarily of hydrophobic amino acid residues). The surface active domain is identified by the positively charged amino acid residues and the hydrophobic opposing surface.

Various computational analyses can be used to determine whether a compound is sufficiently similar to the three-dimensional structure desired. Such analyses can be carried out in current software applications, as known in the art. This can involve a comparison of three-dimensional structure, hydrophobicity, steric bulk, electrostatic properties, bond angles, size or molecular composition, etc. For example, Quanta's Molecular Similarity package (Molecular Simulations Inc., Waltham, Mass.) permits comparison between different structures, different conformations of the same structure, and different parts of the same structure. Typically, the structure of the compound being analyzed is translated and rotated to obtain an optimum fit with the structure of the active polypeptide.

Preferred candidate structures are those having a set of structure coordinates with a root mean square deviation (i.e., the square root of the arithmetic mean of the squares of the deviations of the mean) of conserved residue atoms of less than 2.0 Angstroms when superimposed on the relevant structure coordinates. More preferably, the root mean square deviation is less than 1.0 Angstrom.

In some embodiments, a polypeptide of a modified polypeptide of the present invention may have a beta-sheet structure. For example, a polypeptide of a modified polypeptide of the present invention may be one or more of a series of designed peptide 33-mers referred to as the βpep peptides and known to be bactericidal and capable of neutralizing the bacterial endotoxin lipopolysaccharide (LPS). See Mayo et al., Biochim. Biophys. Acta 1425, 81-92 (1998). These novel beta-sheet-forming peptide 33-mers were designed by using a combination approach employing basic folding principles and incorporating short sequences from the beta-sheet domains of anti-angiogenic proteins.

One of these designed peptides, the βpep-25 peptide having the amino acid sequence ANIKLSVQMKLFKRHLKWKIIVKLNDGRELSLD (SEQ ID NO: 19), also has potent anti-angiogenic activity. See Griffloen et al., Biochem J. 354(Pt 2):233-42 (2001). βpep-25 also inhibits vascular endothelial cell proliferation and induces apoptosis in these cells, as shown by flow-cytometric detection of sub-diploid cells, TUNEL (terminal deoxyribonucleotidyl transferase-mediated dUTP-nick-end labelling) analysis and cell morphology. βpep-25 also inhibits endothelial cell adhesion to and migration on different extracellular matrix components. Inhibition of angiogenesis in vitro was demonstrated in the sprout-formation assay and in vivo in the chick embryo chorio-allantoic membrane angiogenesis assay. See Griffioen et al., Biochem J. 354(Pt 2):233-42 (2001) and WO 01/53335.

A polypeptide of a modified polypeptide of the present invention may be one of a series of dodecapeptides that “walk through” the amino acid sequence of the βpep-25 peptide. Such dodecapeptides include the SC-1 to SC-8 dodecapaptides; the SC-1 dodecapaptide having the amino acid sequence ANIKLSVQMKLF (SEQ ID NO: 1); the SC-2 dodecapaptide having the amino acid sequence KLSVQMKLFKRH (SEQ ID NO:2); the SC-3 dodecapaptide having the amino acid sequence VQMKLFKRHLKW (SEQ ID NO:3); the SC-4 dodecapaptide having the amino acid sequence KLFKRHLKWKII (SEQ ID NO:4); the SC-5 dodecapaptide having the amino acid sequence KRHLKWKIIVKL (SEQ ID NO:5); the SC-6 dodecapaptide having the amino acid sequence LKWKIIVKLNDG (SEQ ID NO:6); the SC-7 dodecapaptide having the amino acid sequence KIIVKLNDGREL (SEQ ID NO:7); and the SC-8 dodecapaptide having the amino acid sequence VKLNDGRELSLD (SEQ ID NO:8). See Mayo et al., Biochem J. 349(Pt 3):717-28 (2000) and WO 01/53335. Thus, in some embodiments of the present invention, the polypeptide of a modified polypeptide may have the amino acid sequence of one or more of the dodecapeptides of SEQ ID NOs: 1-8. In other embodiments, a polypeptide of a modified polypeptide may have an amino acid sequence representing one or more of the following sequences of the βpep-25 peptide; QMKLFKRHLKWK (SEQ ID NO:9), MKLFKRHLKWKI (SEQ ID NO: 10), and/or MKLFKRHLKWKIIV (SEQ ID NO: 11).

The ability of the SC-1 to SC-8 dodecapeptides to kill Gram-negative and Gram-positive bacteria and to neutralize endotoxin is as reported in Mayo et al., Biochem J. 349(Pt 3):717-28 (2000) and WO 01/53335. For all SC peptides, circular dichroic data strongly indicates the presence of both α-helix or 3₁₀-helix. NOESY data acquired on SC peptides in the presence of 30% trifluoroethanol, also show NOEs characteristic of both α-helix or 3₁₀-helix. For the dodecapeptide SC-4 dodecapapetide, which is most 3₁₀ helix-like, NOE-based computational modeling yielded an amphipathic 3₁₀ helical structure in which one surface includes four positively charged amino acid residues and the opposing surface includes hydrophobic amino acid residues. Specifically, the positively charged amino acid residues K1, K4, R5, K8 and K10 are arrayed pentagonally on one face of the helix. More specifically, for SC-4, the surface active domain includes the structure coordinates of the atoms of the amino acid residues K1, K4, R5, and K8, as presented in WO 01/53335.

In some embodiments, a polypeptide of a modified polypeptide may have the amino acid sequence of the SC-4 dodecapeptide, KLFKRHLKWKII (SEQ ID NO:4). The SC-4 dodecapeptide has been identified as a potent antibacterial. The SC-4 dodecapeptide displays bactericidal activity at nanomolar concentrations against Gram-negative bacteria and sub-micromolar concentrations against Gram-positive bacteria. The SC-4 dodecapeptide also effectively neutralizes lipopolysaccharide endotoxin and shows no hemolytic activity below 100 μM. SC-4 folds as an amphipathic helix in membrane-mimicking trifluoroethanol/water solutions and appears to exert bactericidal effects through a membrane-permeabilizing mechanism. Relative to other known bactericidal peptides in the linear peptide, helix-forming catagory, SC-4 appears to be the most potent, broad spectrum bactericidal agent identified to date. See Mayo, et al., Biochem. J. 349, 717-728 (2000) and WO 01/53335.

The polypeptide of the modified polypeptide of the present invention may be one of the single-residue substituted variants of SC-4 that have been previously investigated (Mayo, et al., Biochem. J. 349, 717-728 (2000) and WO 01/53335). Such variants can include lysine/arginine-substituted norleucine variants of SC-4, for example, the substitution of isoleucine for one or more of the K1, K4 and/or R5 positions of SC-4. For example, in some embodiments, a polypeptide may have an amino acid sequence selected from XLFKRHLKWKII (SEQ ID NO: 12); KLFXRHLKWKII (SEQ ID NO: 13); KLFKRHLXWKII (SEQ ID NO: 14); KLFKRHLKWXII (SEQ ID NO: 15); KLFKKHLKWKII (SEQ ID NO: 16); or KLFKHLKWKII (SEQ ID NO: 17), where X is an amino acid, natural or synthetic. Preferably, X is norleucine.

A polypeptide of a modified polypeptide of the present invention may be a polypeptide having an amino acid sequence selected from SEQ ID NOs: 1-17 or an active analogs thereof, wherein X is an amino acid. In some embodiments, X may be norleucine. A polypeptide of a modified polypeptide of the present invention may be a polypeptide having an amino acid sequence selected from SEQ ID NOs: 1-8 or an active analogs thereof.

As used herein, an “active analog thereof” of a polypeptide includes the deletion of one, two, three, or more more contiguous or noncontiguous amino acid residues, the addition of one, two, three, or more more contiguous or noncontiguous amino acid residues, and/or the substitution of one, two, three, or more amino acid residues with a different amino acid residue. Substitutes for an amino acid in the polypeptides of the invention are preferably conservative substitutions, which are selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide.

For the purposes of this invention, conservative amino acid substitutions are defined to result from exchange of amino acids residues from within one of the following classes of residues: Class I: Ala, Gly, Ser, Thr, and Pro (representing small aliphatic side chains and hydroxyl group side chains); Class II: Cys, Ser, Thr, and Tyr (representing side chains including an —OH or —SH group); Class III: Glu, Asp, Asn, and Gln (carboxyl group containing side chains): Class IV: His, Arg, and Lys (representing basic side chains); Class V: Ile, Val, Leu, Phe, and Met (representing hydrophobic side chains); and Class VI: Phe, Trp, Tyr, and His (representing aromatic side chains). The classes also include related amino acids such as 3Hyp and 4Hyp in Class I; homocysteine in Class II; 2-aminoadipic acid, 2-aminopimelic acid, β-carboxyglutamic acid, β-carboxyaspartic acid, and the corresponding amino acid amides in Class III; ornithine, homoarginine, N-methyl lysine, dimethyl lysine, trimethyl lysine, 2,3-diaminopropionic acid, 2,4-diaminobutyric acid, homoarginine, sarcosine and hydroxylysine in Class IV; substituted phenylalanines, norleucine, norvaline, 2-aminooctanoic acid, 2-aminoheptanoic acid, statine and β-valine in Class V; and naphthylalanines, substituted phenylalanines, tetrahydroisoqui noline-3-carboxylic acid, and halogenated tyrosines in Class VI.

Analogs thereof, as used herein, also includes polypeptides modified to include one or more chemical and/or enzymatic derivatizations at one or more constituent amino acid, including, for example, side chain modifications, backbone modifications, and N- and C-terminal modifications including acetylation, hydroxylation, methylation, amidation, and the attachment of carbohydrate or lipid (moieties, cofactors, and the like). Analogs can also include peptidomimetics (e.g., peptidic compounds in which the peptide backbone is substituted with one or more benzodiazepine molecules and polypeptides in which one or more L-amino acids are substituted with the corresponding D-amino acids. Preferred analogs are characterized by having at least one of the biological activities described herein. Such an analog is referred to herein as a “biologically active analog” or simply an “active analog.”

Modified polypeptides of the present invention may vary in length. For example, in some embodiments, a polypeptide may be about 33 amino acids in length. In some embodiments, a polypeptide may be about 8 to about 33 amino acids in length, about 10 to about 25 amino acids in length, about 10 to about 14 amino acids in length or about 12 to about 14 amino acids in length. In some embodiments, a polypeptide may have up to 14 amino acid residues, up to 12 amino acid residues, up to 10 amino acid residues. A polypeptide may be 9 amino acids in length, 10 amino acids in length, 11 amino acids in length, 12 amino acids in length, 13 amino acids in length, 14 amino acids in length, 15 amino acids in length, or 16 amino acids in length. In a preferred embodiment, a polypeptide is a dodecamer, having twelve amino acids residues.

The polypeptides may be synthesized by the solid phase method using standard methods based on either t-butyloxycarbonyl (BOC) or 9-fluorenylmethoxy-carbonyl (FMOC) protecting groups. This methodology is described by G. B. Fields et al. in “Synthetic Peptides: A User's Guide,” W. M. Freeman & Company, New York, N.Y., pp. 77-183 (1992). Polypeptides may also be synthesized via recombinant techniques well known to those skilled in the art. For example, U.S. Pat. No. 5,595,887 describes methods of forming a variety of relatively small peptides through expression of a recombinant gene construct coding for a fusion protein which includes a binding protein and one or more copies of the desired target peptide. After expression, the fusion protein is isolated and cleaved using chemical and/or enzymatic methods to produce the desired target peptide.

### Acylalion

A modified polypeptide of the present invention is a polypeptide that has been modified by fatty acid conjugation. Such a modified polypeptide may also be referred to herein as a peptide-amphiphile, an acylated polypeptide, an acylpeptide conjugate, a lipopeptide, a lipidated peptide, or lipopeptide conjugate.

While native occurring lipopeptides with antibacterial, antifungal, antiviral, or cytolytic activity have been noted (Arima et al., Biochem. Biophys. Res. Commun. 31, 488-494 (1968); Bernheimer et al., J. Gen. Microbiol. 61, 361-369 (1970); Muhlradt et al., J. Exp. Med. 185, 1951-1958 (1997); and Vollenbroich et al., Biologicals 25, 289-297 (1997)), such native lipopeptides differ from the modified polypeptides of the present invention in their unusually short length (usually about 6 to 7 amino acids in length), cyclization (De Lucca et al., Antimicrob. Agents Chemother. 43, 1-11 (1999)), negative charge, and composition of mainly hydrophobic and acidic amino acids.

Modification of a polypeptide by fatty acid conjugation, also referred to herein as acylation with fatty acid residues, fatty acid acylation, or conjugation with lipophilic acids, is by covalent bonding. Such modification may be any of the many methods available to the skilled artisian. For example, fatty acid conjugation can be carried out as set forth in Example 1, on a resin bound peptide using manual Fmoc solid-phase chemistry, essentially as described by Berndt et al., J. Am. Chem. Soc. 117, 9515-9522 (1995). Fatty acid conjugation may also be carried out using the methods used to produce fatty-acid conjugates of cathepsin G peptides (Shafer et al., J. Biol. Chem. 266, 112-116 (1991) and Mak et al., Int. J. Antimicrob. Agents 21, 13-19 (2003)), lactoferrin peptides (Wakabayashi et al., Antimicrob. Agents Chemother. 43, 1267-1269 (1999) and Majerle et al., J. Antimicrob. Chemother. 51, 1159-1165 (2003)), magainin paptides (Avrahami and Shai, Biochemistry 41, 2254-2263 (2002)) or cercropin-melittin peptides (Chicharro et al., Antimicrob. Agents Chemother. 45, 2441-2449 (2001)).

In the modified polypeptide of the present invention, fatty acid conjugates may be positioned at the N-terminus of a polypeptide, the C-terminus of a polypeptide, and/or internally within the sequence of the polypeptide. Preferably, such modification may be bonded to the polypeptide at the N-terminus and/or the C-terminus, more preferably at the N-terminus.

Modified polypeptides of the present invention include polypeptides that have been modified to include a linear or branched aliphatic group having at least 6 carbon atoms. In some embodiments the linear or branched aliphatic (preferably alkyl) group may have about 8 to about 22 carbon atoms. In some embodiments the aliphatic group may have about 10 to about 20 carbon atoms. In some embodiments the aliphatic group may have about 12 to about 18 carbon atoms. In some embodiments the linear or branched aliphatic group may have more than 22 carbon atoms. The linear or branched aliphatic group may include one or more unsaturated carbon-carbon bonds. These can be double or triple carbon-carbon bonds, but typically, if they are present, they are double bonds. If present, there are typically only one or two unsaturated carbon-carbon bonds.

The linear or branched aliphatic group may be derived from a fatty acid. When a fatty acid is used to modify a polypeptide, one of the carbon atoms of the modification will be from the carbonyl carbon of the fatty acid. When an aliphatic group or alkyl group other than a fatty acid is used to modify a polypeptide, the number of carbon atoms will be one less, as there is no contribution of a carbonyl carbon from the fatty acid. Fatty acids include carboxylic acids derived from or contained in an animal or vegetable fat or oil. Fatty acids are composed of a hydrocarbon chain containing from 4 to 22 carbon atoms (usually even-numbered) and characterized by a terminal carboxyl group—COOH. For example, a fatty acid with 8 to 22 carbond atoms (C8 to C22) may be used in the modified polypeptides of the present invention. Preferably, a C10 to C20 fatty acid may be used. The generic formula for the above acetic acid is CH₃ (CH₂)ₓCOOH (the carbon atom count includes the carboxyl group). Fatty acids may be saturated or unsaturated (i.e., olefinic), and either solid, semisolid, or liquid. They are classified among the lipids together with soap and waxes. A saturated fatty acid is a fatty acid in which the carbon atoms of the alkyl chain are connected by single bonds. Common saturated fatty acids include butyric (C₄), lauric (C₁₂), palmitic (C₁₈), and stearic (C₁₈). An unsaturated fatty acid is a fatty acid in which there are one or more double or triple bonds between the carbon atoms in the chain. These acids are usually vegetable-derived and consist of carbon chains containing 18 or more carbon atoms with the characteristic end group —COOH.

In some embodiments, the aliphatic group includes one or more unsaturated carbon-carbon bonds. In some embodiments, the linear or branched aliphhatic group is an alkyl group derived from a fatty acid. The fatty acid may be a C8-C22 fatty acid. The fatty acid may be a C10-C20 fatty acid. The fatty acid may be a C10-C20 fatty acid, wherein the linear or branched alkyl group has at least 11 carbon atoms, and wherein the linear or branched alkyl group has 11 to 19 carbon atoms.

### Antibacterial Activity

Modified polypeptides of the present invention demonstrate antibacterial activity. Bactericidal activity can be evaluated against a variety of bacteria, including Gram-negative bacteria or Gram-positive bacteria. The types of bacteria can include Pseudomonas spp, including P. aeruginosa and P. cepacia, E. coli strains, including E. coli B, Salmonella, Proteus mirabilis and Staphylococcus strains such as Staphylococcus aureus. Modified polypeptides of the present invention may demonstrate antibacterial activity against clinically-relevant, drug-resistant strains of bacteria.

The modified polypeptides of the present invention can be added to cells in culture or used to treat a subject, such as a mammalian subject, including a human subject. Where the modified polypeptides are used to treat a subject, the modified polypeptide may be in composition along with a pharmaceutically acceptible carrier and/or pharmaceutically acceptible buffer. Treatment can be prophylactic or therapeutic. Thus, treatment can be initiated before, during, or after the development of the condition to be treated, such as bacterial infection and/or endotoxemia. As such, the phrases “inhibition of or “effective to inhibit” a condition such as a bacterial infection and/or endotoxemia, for example, includes both prophylactic and therapeutic treatment (i.e., prevention and/or reversal of the condition).

The present invention provides a method for treating a bacterial infection in a subject by administering to a subject one or more modified polypeptides described herein effective in an amount effective to inhibit the bacterial infection. The present invention also provides a method for inhibiting bacterial infection in vitro by contacting cells with one or more of the modified polypeptides described herein in an amount effective to inhibit the bacterial infection, inhibit bacterial cell growth, and/or demonstrate bactericidal activity.

The effective amount of a peptide for treating a bacterial infection will depend on the bacterial infection, the location of the infection and the peptide. An effective amount of the peptide for treating bacterial infection is that amount that diminishes the number of bacteria in the animal and that diminishes the symptoms associated with bacterial infection such as fever, pain and other associated symptoms of the bacterial infection. The effective amount of a peptide can be determined by standard dose response methods in vitro and an amount of peptide that is effective to kill at least about 50% to about 100% of the bacteria (LD₅₀) and more preferably about 60% to about 100% of the bacteria would be considered an effective amount. Preferably, the peptide has an effective dose at a concentration of about 1×10⁻⁴ M to about 1×10⁻¹⁰ M, and more preferably at a concentration of about 1×10⁻⁷M to about 1×10⁻⁹M. Peptides that are considered to be bactericidal may kill at least one organism selected from the group of P. aeruginosa, P. cepacia, E. coli B, Salmonella, Proteus mirabilis, and Staphylococcus aureus at concentrations of about 10⁻¹⁰ M or greater under physiological conditions (e.g., at about pH of 7.4

Alternatively, an effective amount of the modified polypeptide for treating a bacterial infection can be determined in an animal system such as a mouse. Acute peritonitis can be induced in mice such as outbred Swiss webster mice by intraperitoneal injection with bacteria such as P. aeruginosa as described, for example, by Dunn et al. (Surgery, 98:283, 1985) or Cody et al. (Int. Surg. Res., 52:315, 1992). Different amounts of peptide can be injected at one hour intravenously prior to the injection of the bacteria. The percentage of viable bacteria in blood, spleen, and liver can be determined in the presence and absence of the peptide or other antibiotics. While not meant to limit the invention, it is believed that bactericidal peptide could also enhance the effectiveness of other antibiotics such as erythromycin, and the like.

In both in vivo and in vitro methods, “inhibiting” a bacterial infection includes preventing as well as reversing or reducing the growth of bacteria in a subject or a cellular sample. The level of bacterial infection can be determined according, for example, to the bactericidal assays described in the Examples Section. These assays can be used to determine the effectiveness of a polypeptide, whether used in vivo or in vitro. To determine the effectiveness of the treatment of a patient having a bacterial infection, a blood sample can be taken, a culture developed, and the amount of live bacteria determined according to the bactericidal assay described in the Examples Section.

One or more modified polypeptides with bactericidal activity can be combined with other agents that are known and used to treat bacterial infections.

The modified polypeptides of the present invention also demonstrate endotoxin neutralizing activity and may be used to treat endotoxemia. The present invention provides a method for treating endotoxemia in a subjet. This involves administering to a subject one or more modified polypeptides described herein in an amount effective to neutralize endotoxin. The present invention also provides a method for neutralizing endotoxin in vitro by contacting cells with one or more of the modified polypeptides described herein in an amount effective to neutralize endotoxin.

Endotoxemia is typically caused by toxic LPS from Gram-negative bacteria, such as Pseudomonas spp., rough strains of E. coli, encapsulated E. coli and smooth strain E. coli, but can also be caused by Gram-positive bacteria, and occasionally, by fungi. Components released by Gram-positive bacteria that can cause endotoxemia include peptidoglycan and lipoteichoic acid, and lipoarabinomannan from the cell wall of Mycobacterium spp. Animals systemically infected with Gram-negative bacteria exhibit symptoms of endotoxin shock (also referred to as endotoxic shock, septic shock, circulatory shock, and septicemia) such as fever, shock, and TNF-α release.

Activation of a cell by a toxic LPS-containing complex results in the synthesis, release, or activation of cell-derived proinflammatory mediators, which can include cytokines (such as interleukin-1, interleukin-6, interleukin-8, and tumor necrosis factor α), platelet activating factor, nitric oxide, complement (e.g., C5a and C3a), prostagladins, leukotrienes, the kinin system, oxygen metabolites, catecholamines and endorphines. The mediators can impact organ systems including the heart, vascular system, coagulation system, lungs, liver, kidney and the central nervous system.

Endotoxin neutralizing activity can be measured by determining the molar concentration at which the modified polypeptide completely inhibits the action of lipopolysaccharide in an assay such as the Limulus amoebocyte lysate assay (LAL, Sigma Chemicals, St. Louis, Mo.) or the chromogenic LAL 1000 test (Biowhittacker, Walkersville, Md.). Endotoxin neutralizing activity can also be measured by calculating an inhibitory dose 50 (LD₅₀) using standard dose response methods. An inhibitory dose 50 is that amount of peptide that can inhibit 50% of the activity of endotoxin. Peptides preferably neutralized endotoxin at a molar concentration of about 1×10⁻⁴ M to about 10⁻⁸ M, more preferably about 10⁻⁵ M to about 10⁻⁶ M. Peptides considered to not have endotoxin neutralizing activity do not neutralize endotoxin at a molar concentration of about 10⁻⁴ M or less.

In both the in vivo and in vitro methods, “neutralizing” endotoxin includes binding LPS and thereby removing it from the system of a subject or a cellular sample. The level of endotoxemia can be determined, for example, according to the LPS neutralization assay described in the Examples Section. These assays can be used to determine the effectiveness of a polypeptide, whether used in vivo or in vitro. To determine the effectiveness of the treatment of a subject having endotoxemia, a blood sample can be taken, a culture developed, and the amount of cytokines (e.g., TNF-α, IL-1) can be determined using methods known to one of skill in the art. For example, the WEHI assay can be used for the detection of TNF-α (Battafarano et al., Surgery 118, 318-324 (1995)).

One or more modified polypeptides with endotoxin neutralizing can be combined with other agents that are known and used to treat endotoxin shock. Studies have shown that serum concentrations of the cytokine tumor necrosis factor (TNF) increase after onset of endotoxemia, and serum TNF activity is directly associated with the onset of signs of abdominal pain and fever, for example. Thus, endotoxin activity can also be measured by determining the amount of release of tumor necrosis factor alpha (TNF-α) from a macrophage cell line or by evaluating the symptoms of shock in animals. Production of TNF-α can be assayed as described by Mossman et al.(Immunological Methods 65:55, 1983).

The modified polypepetides of the present invention may be used in methods for decreasing the amount of TNFα in a subject. This involves administering to a subject one or more of the modified polypeptides described herein in an amount to decrease the amount of TNFα in a subject's system as determined by evaluating serum levels of TNFα. Further, the present invention provides a method for decreasing the amount of TNFα in vitro by contacting and/or incubating cells with one or more of the modified polypeptides described herein in an amount effective to decrease TNFα amounts in the cell culture. For both in vivo and in vitro methods, the WEHI assay can be used for the detection of TNFα (Battafarano et al., Surgery 118, 318-324 (1995)) in cell culture or in serum from a patient. Alternatively, the amount of TNFα in a sample can be assayed using an anti-TNFα antibody. A modified polypeptide “active” for decreasing TNFα can be evaluated using an in vitro test, and preferably shows an at least 10% decrease in the amount of TNFα.

Modified polypeptides of the present invention may demonstrate antifungal activity and may be used to treat fungal infections. The present invention provides a method for treating fungal infections in a subject. This involves administering to a subject one or more of the modified polypeptides described herein in an amount effective to inhibit fungal growth. Further, the present invention provides a method for inhibiting fungal growth in vitro by contacting cells with one or more of the modified polypeptides described herein in an amount to inhibit fungal cell growth. The antifungal activity of a modified polypeptide may be assayed, for example, as described in Cavallarin et al., Mol Plant Microbe Interact. 11(3), 218-27 (1998).

Modified polypeptides of the present invention may demonstrate antiparasitic activity and may be used to treat parasitic infections. The present invention provides a method for treating parasitic infections in a subject. This involves administering to a subject one or more modified polypeptides described herein in an amount to inhibit parasitic activity. The present invention also provides a method for inhibiting parasitic activity in vitro by contacting parasites and/or cells infected with a parasite with one or more modified polypeptides described herein in an amount effective to inhibit parasitic metablism, growth, and/or replication. The antiparasitic activity of a modified polypeptide may be assayed, for example, as described in Chicarro et al., Antimicrob Agents Chemother 45(9), 2441-9 (2001).

Angiogenesis is crucial to numerous biological functions in the body, from normal processes like embryogenesis and wound healing to abnormal processes like tumor growth, arthritis, restenosis and diabetic retinopathy and the use of agents that inhibit angiogenesis in vitro and in vivo will be an effective therapeutic modality, particularly in the treatment of tumors. It has also been postulated that tumor growth can be controlled by deprivation of vascularization (Folkman J. natl. Cancer. Inst. 82, 4-6 (1990); Folkman et al., J. Biol. Chem. 267, 10931-10934 (1992)). A growing number of endogenous inhibitors of angiogenesis such as platelet factor-4 (PF4), interferon-γ inducible protein-10 (IP-10), thrombospondin-1 (TSP-1), angiostatin, as well as synthetic agents, e.g., thalidomide, TNP-470, and metalloproteinase inhibitors have been described. Some of these agents are currently being tested in phase I/II clinical trials. Previous research described in Griffioen et al., Blood 88, 667-673 (1996), and Griffioen et al., Cancer Res. 56, 1111-1117 (1996) has shown that proangiogenic factors in tumors induce down-regulation of adhesion molecules on endothelial cells in the tumor vasculature and induce energy to inflammatory signals such as tumor necrosis factor α (TNFα), interleukin-1, and interferon-γ. EC exposed to vascular endothelial cell growth factor (VEGF) (Griffioen et al., Blood 88, 667-673 (1996)) and basic fibroblast growth factor (bFGF) (Griffioen et al., Blood 88, 667-673 (1996); and Melder et al., Nature Med. 2, 992-997 (1996)) have a severely hampered up-regulation of intercellular adhesion molecule-1 (ICAM-1) and induction of vascular cell adhesion molecule-1 (VCAM-1) and E-selectin. This phenomenon, which was named tumor-induced EC anergy, is one way in which tumors with an angiogenic phenotype may escape infiltration by cytotoxic leukocytes.

Because angiogenesis-mediated down-regulation of endothelial adhesion molecules (EAM) may promote tumor outgrowth by avoiding the immune response (Griffioen et al., Blood 88, 667-673 (1996); Kitayama et al., Cancer. Res. 54 4729-4733 (1994); and Piali et al., J. exp. Med. 181, 811-816 (1995)), it is believed that inhibition of angiogenesis would overcome the down-regulation of adhesion molecules and the unresponsiveness to inflammatory signals. In support of this hypothesis, a relation between E-selectin up-regulation and the angiostatic agent AGM-1470 has been reported (Budson et al., Biochem. Biophys. Res. Comm. 225, 141-145 (1996)). It has also been shown that inhibition of angiogenesis by PF4 up-regulates ICAM-1 on bFGF-simulated EC. In addition, inhibition of angio-genesis by PF4 overcomes the angiogenesis-associated EC anergy to inflammatory signals.

The present invention provides a method for inhibiting endothelial cell proliferation in a subject by administering to a subject one or more of the modified polypeptides described herein in an amount effective to inhibit the growth of endothelial cells. The present invention also provides a method for inhibiting endothelial cell proliferation in vitro by contacting cells with one or more of the modified polypeptides described herein in an amount effective to prevent and/or reduce the growth of endothelial cells. For determining endothelial cell proliferation, various methods known to one of skill in the art could be used. For example, for evaluation of endothelial cell growth in tumors, tissue sections can be appropriately stained to quantify vessel density. For determining the amount of endothelial cell proliferation in vitro, an EC Proliferation Assay can be used, which involves the uptake of tritiated thymidine by cells in cell culture. A polypeptide that is active for inhibiting endothelial cell proliferation is preferably one that causes at least a 10% reduction in endothelial cell proliferation at a concentration lower than 10⁻⁴ M.

The present invention also provides a method for inhibiting angiogenic-factor mediated inter-cellular adhesion molecule expression down-regulation in a subject by administering to a subject one or more of the modified polypeptides described herein in an amount effective to prevent and/or reduce the amount of ICAM expression down-regulation. The present invention also provides a method for inhibiting angiogenic-factor mediated inter-cellular adhesion molecule expression down-regulation in vitro by contacting cells with one or more of the modified polypeptides described herein in an amount effective to prevent and/or reduce the amount of ICAM expression down-regulation.

The present invention provides a method for inhibiting angiogenesis in a subject by administering to a subject one or more of the modified polypeptides described herein in an amount effective to prevent and/or reduce angiogenesis. The present invention also provides a method for inhibiting angiogenesis in vitro by contacting cells with one or more of the modified polypeptides described herein in an amount effective to prevent and/or reduce angiogenesis, wherein the composition includes. For determining the amount of angiogenesis in vivo, various methods known to one of skill in the art could be used. For example, for evaluation of angiogenesis in tumors, tissue sections can be appropriately stained to quantify vessel density. For determining the amount of angiogenesis in vitro, an in vitro angiogenesis assay can be used, which involves the disappearance of EC sprouting in cell culture. A polypeptide that is “active” for angiogenesis inhibition is preferably one that causes an at least 10% reduction in endothelial cell sprouting at a concentration lower than 10⁻⁴ M.

The present invention provides a method for inhibiting tumorigenesis in a subject by administering to a subject one or more of the modified polypeptides as described herein in an amount effective to prevent and/or reduce tumor growth. Methods of determining the inhibition of tumorigenesis are well known to those of skill in the art, including evaluation of tumor shrinkage, survival, etc.

The methods of the invention include administering to a subject, preferably a mammal, and more preferably a human, the composition of the invention in an amount effective to produce the desired effect. The modified polypeptides can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The modified polypeptides of the present invention may be formulated in pharmaceutical compositions and then, in accordance with the methods of the invention, administered to a subject, in a variety of forms adapted to the chosen route of administration. The formulations may be conveniently presented in unit dosage form and may be prepared by any of the methods well known in the art of pharmacy. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations suitable for parenteral administration conveniently include a sterile aqueous preparation of the active agent, or dispersions of sterile powders of the active agent, which are preferably isotonic with the blood of the recipient. Isotonic agents that can be included in the liquid preparation include sugars, buffers, and sodium chloride. Solutions of the active agent can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder or granules, as liposomes containing the chemopreventive agent, or as a solution or suspension in an aqueous liquor or non-aqueous liquid such as a syrup, an elixir, an emulsion, or a draught. Such compositions and preparations typically contain at least about 0.1 weight percent of the active agent. The amount of polypeptide (i.e., active agent) is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Actylation of SC4 Dodecapeptide Increases Bactericidal Potency Against Gram-Positive and Drug-Resistant Bacteria

Dodecyl and octadecyl fatty acids were conjugated to the N-terminus of SC4, a potently bactericidal, helix-forming peptide 12-mer with the amino acid sequence KLFKRHLKWKII (SEQ ID NO:4) and the bactericidal activities of the resultant SC4 “peptide-amphiphile” molecules examined. SC4 peptide-amphiphiles showed up to a thirty fold increase in bactericidal activity against Gram-positive strains S. aureus, S. pyogenes, and B. anthracis, including S. aureus strains resistant to conventional antibiotics, but little or no increase against the Gram-negative bacteria E. coli and P. aeruginosa. Fatty acid conjugation improved endotoxin (lipopolysaccharide) neutralization three to six fold. Although acylation somewhat increased lysis of human erythrocytes, it did not increase lysis of endothelial cells, and the hemolytic effects occurred at concentrations 10- to 100-fold higher than those required for bacterial cell lysis. For insight into the mechanism of action of SC4 peptide-amphiphiles, circular dichroism, NMR, and fluorescence spectroscopy studies were performed in micelle and liposome models off eukaryotic and bacterial cell membranes. Circular dichroism indicated that SC4 peptide-amphiphiles had the strongest helical tendencies in liposomes mimicking bacterial membranes, and strong membrane integration of the SC4 peptide-amphiphiles was observed with tryptophan fluorescence spectroscopy under these conditions; results that correlated with the increased bactericidal activities of SC4 peptide-amphiphiles. NMR structural analysis in micelles demonstrated that the two-thirds of the peptide closest to the fatty-acid tail exhibited a helical conformation, with the positively-charged side of the amphipathic helix interacting more with the model membrane surface. These results show that conjugation of a hydrophobic fatty acid to the SC4 peptide increases bactericidal potency by enhancing membrane interactions and stabilizing helical structure of the peptide in the membrane-bound state.

### MATERIALS AND METHODS

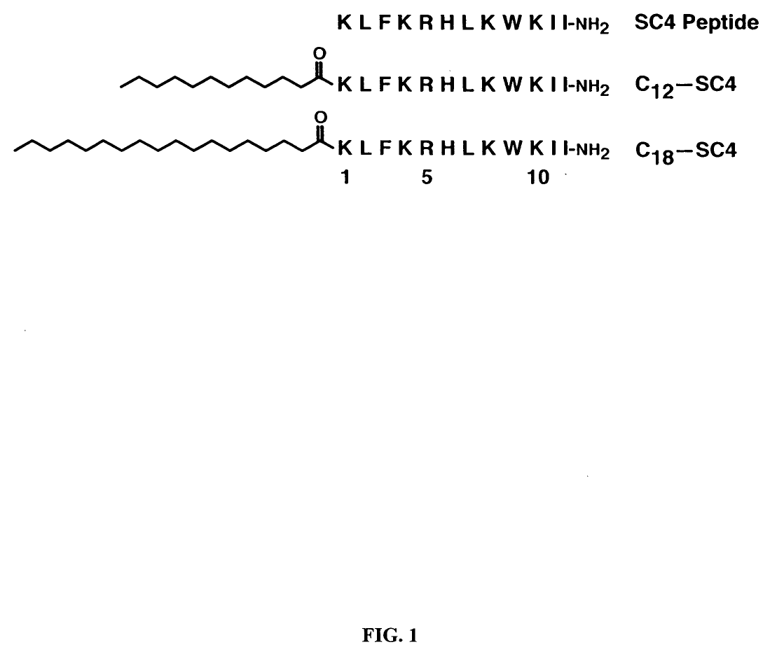

Peptide sequences and amphiphile structure. The designed, 12-residue SC4 peptide sequence was based on a peptide derived from bactericidal/permeability-increasing protein (Mayo et al., Biochem. J. 349, 717-728 (2000)). C12 and C18 amphiphile versions of SC4 had the appropriate fatty acid covalently, linked to the N-terminus of the peptide (FIG. 1). The SC4 peptide and the SC4 peptide-amphiphiles were amidated at the C-terminus.

SC4 peptide synthesis. The SC4 peptide was synthesized at the University of Minnesota Microchemical Facility on a Milligen/Biosearch 9600 peptide solid-phase synthesizer using 9-fluorenylmethyloxycarbonyl (Fmoc) chemistry. Lyophilized crude peptide was purified by preparative reversed-phase high performance liquid chromatography (HPLC) on a C18 column with an elution gradient of 0-60% acetonitrile with 0.1% trifluoroacetic acid in water. Purity and composition of the peptide was verified by HPLC, amino acid analysis, and matrix-assisted laser desorption/ionization time-of-flight (MALDI-TOF) mass spectrometry.

SC4 peptide-amphiphile synthesis. Peptide-amphiphiles were synthesized from resin-bound SC4 peptide and dodecanoic (lauric) or octadecanoic (stearic) fatty acids with manual Fmoc solid-phase chemistry essentially as described previously (Berndt et al., J. Am. Chem. Soc. 117, 9515-9522 (1995)). Briefly, the C12 or C18 fatty acid tails were N-terminally coupled to the resin-bound peptide for three hours with four fold molar excess of 2-(1H-benzotriazole-1-yl)-1,1,3,3-tetramethyluronium hexafluorophosphate (HBTU), 1-hydroxybenzotriazole (HOBt), N,N-diisopropylethylamine (DIEA), and fatty acid tails in dichloromethane (DCM)/N,N-dimethylformamide (DMF). Peptide-amphiphiles and protecting groups were cleaved from the resin by treatment with Reagent K (82.5% trifluoroacetic acid (TFA), 5% phenol, 5% H₂O, 5% thioanisole, 2.5% ethanedithiol) for two hours. Crude peptide-amphiphiles were purified by HPLC on a reversed-phase C4 column with a gradient of 30-60% (for C12-SC4) or 35-70% (for C18-SC4) acetonitrile in water with 0.1% TFA. The identity of the purified peptide-amphiphile products was verified by MALDI-TOF mass spectrometry.

Bacterial strains. Gram-negative Escherichia coli J96 and IA2 are smooth strain, uropathogenic clinical isolates described by Johnson and Brown (Johnson and Brown, J. Infect. Dis. 173, 920-926 (1996)). Pseudomonas aeruginosa type I is a clinical smooth-strain isolate serotyped by using the scheme of Homma et al., Japan. J. Exp. Med. 46, 329-336 (1976) and maintained in the lab by monthly transfer on blood agar plates. Gram-positive MN8 and MNHO are patient isolates of Staphylococcus aureus; Eaton and Wilson are isolates of Streptococcus pyogenes from two patients (Lockwood et al., Biochem. J. 378, 93-103 (2004). M497880 and W73134 are clinical isolate strains of S. aureus that display resistance to all conventional antibiotics except vancomycin (Lockwood et al., Biochem. J. 378, 93-103 (2004). Bacillus antracis is a laboratory strain from the lab of P. M. Schlievert. All Gram-positive strains were provided by P. M. Schlievert. E. coli and S. aureus strains were maintained and plated on nutrient agar plates. S. pyogenes strains were maintained on blood agar plates and plated on brain-heart infusion agar plates. B. anthracis was grown in Todd Hewitt broth.

Bactericidal assay. Pyrogen-free solutions were used throughout the assay. Log phase bacteria were obtained by transferring an overnight culture or scraping crystals from −85° C. glycerol stocks of overnight cultures. Bacteria were washed and resuspended in 0.9% sodium chloride solution with adjustment to an optical density at 650 nm that yielded 3×10⁸ colony-forming units (CFU)/milliliter (mL). Bacteria were then diluted 1:10 in 0.08 M citrate phosphate buffer, pH 7.0 (prepared by mixing 0.08 M citric acid with 0.08 M dibasic sodium phosphate). Bacteria (0.15 mL) were combined with the appropriate amount of peptide and 1.0 mL buffer in 17×100 mm polypropylene tubes and incubated in a reciprocal water bath shaker at 37° C. for 30 minutes. For all bacterial strains, except B. anthracis, 1:10, 1:100, and 1:1000 dilutions were then prepared in 0.9% sodium chloride solution and 20 microliters (μL or μl) of each dilution was streaked across an agar plate. Gram-negative organisms were plated on nutrient agar plates containing 2% agar and Gram-positive organisms were plated on MacConkey agar (2%). Plates were incubated overnight at 37° C. and colonies counted the next morning. The dilution containing 10-100 bacterial colonies was counted and the number multiplied by 50 to adjust all counts to the number bacteria killed per milliliter. For B. anthracis, 5 mL of Todd Hewitt broth was added to the solutions following incubation, and bacteria were grown to mid-log phase under continuous shaking. Cell survival was assayed by optical density at 650 nm for B. anthracis.

Bactericidal activities are reported as LD₅₀ (dose lethal to 50% of bacteria) values determined by fits of the data to the sigmoidal dose-response equation:
% Killed=Rₘᵢₙ+{(Rₘᵢₙ−Rₘₐₓ)/[1+(C/LD₅₀)\hat{ }m]}
where C is the concentration, Rₘᵢₙ=0, the minimum response; Rₘₐₓ=100, the maximum response; LD₅₀ is the midpoint of the transition; and m is the slope of the transition. LD₅₀ and m were the free variables used to fit the data.

Limulus amoebocyte lysate assay for lipopolysaccharide (LPS) neutralization. The ability of SC4 peptide and amphiphiles to neutralize endotoxin was measured using the chromogenic QCL-1000 kit from BioWhittaker, Inc. (Walkersville, Md). This method is quantitative for Gram-negative bacterial endotoxin (LPS) and uses peptide inhibition of LPS-mediated activation of a proenzyme as a measure of activity (Young et al., J. Clin. Invest. 51, 1790-1797 (1972)). Peptides of the appropriate concentration were mixed with Limulus amoebocyte lysate, 0.04 unit (0.01 nanogram (ng)) of E. coli 055:B5 LPS (SIGMA), and a colorless synthetic substrate (Ac-Ile-Glu-Ala-Arg-pNA) (SEQ ID NO: 18). Peptide binding to LPS was determined by monitoring enzymatic conversion of the substrate to yellow p-nitroaniline (pNA) via absorption at 410 nm. Endotoxin concentration was determined from the initial rate of enzyme activation. IC₅₀ (concentration displaying 50% inhibition) values for LPS binding were determined by fitting to a dose-response curve.

Eukaryotic cell lysis activity. The lytic activity of SC4 molecules was tested against human red blood cells and human endothelial cells. Red blood cells were washed three times with phosphate buffered saline (PBS; 35 mM phosphate buffer, 0.15 M NaCl, pH 7.0) prior to performing the hemolysis assay. 100 microliters (μl) of serially-diluted peptides (1-100 micromolar (μM)) in PBS was added to Eppendorf tubes containing 100 μl of 0.4% volume/volume (v/v) human red blood cells suspended in PBS. Tubes were incubated for 1 hour at 37° C. and then centrifuged at 1000×g for 5 minutes. 100 μl aliquots of the supernatant were then transferred to Eppendorf tubes and hemolysis was measured by absorbance at 414 nm. Zero and 100% hemolysis were determined in PBS and 1% Triton-X 100, respectively. The hemolysis percentage was calculated as:
% hemolysis={[(A₄₁₄(peptide)−A₄₁₄(PBS)]/[A₄₁₄(Triton-X 100)−A₄₁₄(PBS)]}×100

Bacterial and eukaryotic cell membrane mimics. Bilayer-forming phospholipids and micelle-forming detergents were used as a membrane mimics. Liposomes formed from zwitterionic 1,2-dipalmitoyl-sn-glycero-3-phosphocholine (DPPC) were used as a mimic of eukaryotic membranes, which are composed of predominantly neutral, zwitterionic lipids. Liposomes composed of a 7:3 molar mixture of neutral DPPE and negatively-charged 1,2-dipalmitoyl-sn-glycero-3-[phospho-rac-(1-glycerol)] (DPPG) were used to mimic bacterial membranes, which have an overall negative membrane charge and a composition essentially matching that used in the mimic. Micelle-forming dodecylphosphatidylcholine (DPC) and sodium dodecyl sulfate (SDS) were used as simple mimics of eukaryotic and bacterial membranes, respectively, in nuclear magnetic resonance (NMR) experiments, which require small aggregate size to limit resonance broadening, and in circular dichroism (CD) experiments in order to minimize light scattering from aggregates.

Liposome preparation. 1,2-dipalmitoyl-sn-glycero-3-phosphocholine (DPPC), 1,2-dipalmitoyl-sn-glycero-3-phosphoethanolamine (DPPE), and 1,2-dipalmitoyl-sn-glycero-3-[phospho-rac-(1-glycerol)] (DPPG) phospholipids were obtained from Avanti Polar Lipids, Inc. Chloroform solutions of pure phospholipid were mixed in a glass tube at desired lipid ratios and dried under a low flow of N₂ to form a thin lipid film. Residual solvent was removed under vacuum for several hours. The resulting lipid film was hydrated at least 1.5 hours at temperatures above the lipid transition temperature with an appropriate volume of water to yield a final lipid concentration of 4 mM. The solutions were periodically vortexed during the hydration period. Solutions were then sonicated 20 minutes or more in a bath sonicator at temperatures above the lipid transition temperature to produce small liposomes. Lipid solutions were cooled to room temperature prior to use.

Liposome and micelle solution preparation. Aqueous stock solutions were prepared by dissolving dry peptide, amphiphile, or detergent in water; aqueous lipid stock solutions were prepared as described above. SDS or DPC detergent-peptide solutions were prepared by separately diluting stock solutions of peptide/amphiphile and detergent with water, to give twice the desired final concentrations. The diluted solutions were mixed to give the appropriate final peptide concentration (0.1 mM for CD, 0.5 mM for NMR) at a peptide: detergent ratio of 1:50 (DPC) or 1:100 (SDS). These ratios correspond to roughly one peptide per micelle, based on the aggregation numbers of the detergents. Detergent micelle solutions for fluorescence spectroscopy were prepared identically but with a final peptide concentration of 5-10 micromolar (μM) and a, peptide: detergent ratio of 1:500 in DPC and 1:1000 in SDS. Liposome solutions were prepared similarly, mixing diluted stocks of peptide and liposomes to give a final peptide concentration of 5-10 μM at a peptide:lipid ratio of 1:20. The method of mixing dilute solutions of peptide and detergent/lipid limited aggregation observed when mixing stocks of higher concentration.

Circular dichroism. CD spectra were recorded on a Jasco J-710 spectrophotometer at 25° C. or 37° C. in a 0.1 cm (aqueous and detergent solutions) or 1.0 cm (lipid solutions) pathlength quartz cuvette. Acquisition was performed with a 50 nm/minute scan rate, 1 nm bandwidth, and 2 second response. The corresponding baseline (water, detergent, or lipid solution) was subtracted from each spectrum (θ). Reported spectra are averages of six scans and are expressed as mean residue ellipticity. Peptide concentrations were 0.1 mM in water or detergent micelle solutions, 10 μM in liposome solutions. CD basis spectra were measured with poly(lysine) and poly(glutamic acid) (Sigma) with conditions and parameters reported by others (Adler et al., Methods Enzymol. 27, 675-735 (1973) and Greenfield et al., Biochemistry 8, 4108-4116 (1969)). Linear combinations of a-helix, β-sheet, and random coil basis spectra were used to fit experimental CD spectra for estimation of secondary structure contributions.

NMR measurements. Solutions for NMR measurements were prepared as described above, but dissolved in 90% H₂0, 10% D₂0 to give a final peptide/amphiphile concentration of 0.5 mM, pH 5.3 (unbuffered). Proton NMR spectra were acquired on a Varian UNITY Plus-600 NMR spectrometer at 25° C. Spin systems were identified with 2D-homonuclear magnetization transfer TOCSY spectra obtained with a mixing time of 60 milliseconds (ms). Nuclear Overhauser effect spectroscopy (NOESY) experiments with a mixing time of 100 ms were performed for conformational analysis. The water resonance was suppressed with WATERGATE total correlation spectroscopy (TOCSY) or WET nuclear Overhauser effect spectroscopy (NOESY) pulse sequences. For structural analysis experiments, 2D-NMR spectra were collected as to 512 tl increments, each with 1k complex data points over a spectral width of 8 kHz in both dimensions with the carrier placed on the water resonance. Thirty-two scans were averaged for each t1 increment. Data were processed offline with NMRPipe (Delaglio et al., J. Biomol. NMR 6, 277-293 (1995)) and Sparky (provided by Goddard, T. D. and Kneller, D. G., University of California, San Francisco) on an Apple iBook. Data sets were multiplied in both dimensions by a shifted sine-bell function, baseline-corrected, and zero-filled to 1k in the t1 dimension and 2k in the t2 dimension prior to Fourier transformation. Shorter TOCSY experiments (4 transients for each of 128 t1 increments) were used to investigate peptide-micelle interactions and for examining hydrogen-bonding through the temperature dependence of NH chemical shifts.

Structural modeling. Inter-proton distance constraints were derived from nuclear Overhauser effect (NOE) crosspeaks assigned in ¹H NOESY spectra. NOEs were classified as strong, medium, weak or very weak corresponding to upper-bound distance constraints of 2.9, 3.3, 4.0, and 5.0 Å, respectively. The lower-bound constraint between non-bonded protons was set to 1.8 Å. A 0.5 Å correction was added to the upper bound for NOEs involving side-chain protons. Hydrogen bond constraints were identified from the pattern of sequential and interstrand NOEs involving NH and CₐH protons, together with evidence of slow amide proton-solvent exchange. Each hydrogen bond was defined by upper-bound distance constraints of 3.5 Å and 4.0 Å for NH-O and N-O distances, respectively.

The X-PLOR software package (Nilges, M., Kuszewski, J. and Brunger, A. T. (1991) in Computational Aspects of the Study of Biological Macromolecules by NMR, Plenum Press, New York ) was used with NOE-derived distance constraints to calculate structures of C12-SC4 in DPC micelles following a method described previously (Mayo et al., Biochem. J. 349, 717-728 (2000)). Final structures were obtained by filtering the results such that none of the final structures had NOE violations greater than 0.5 Å, and the bond angles, lengths or improper angles, did not deviate from ideal geometry more than 5°, 0.05 Å, and 5°, respectively. Structures were superimposed and visualized with the VMD software package (Humphrey et al., J. Mol. Graph. 14, 33-38 (1996)) and analyzed with X-PLOR routines.

Tryptophan fluorescence spectroscopy. Tryptophan fluorescence spectra were obtained at 25 or 37° C. on an ISS-K2 steady-state fluorometer. Solutions were prepared as described above and placed in a I-cm quartz cuvette for measurement. Peptide concentration was 5 μM in water or 1:20 (mol/mol) lipid solutions. Samples were excited at 280 nm and emission recorded from 300-450 nm at a resolution of 1 nm.

### RESULTS

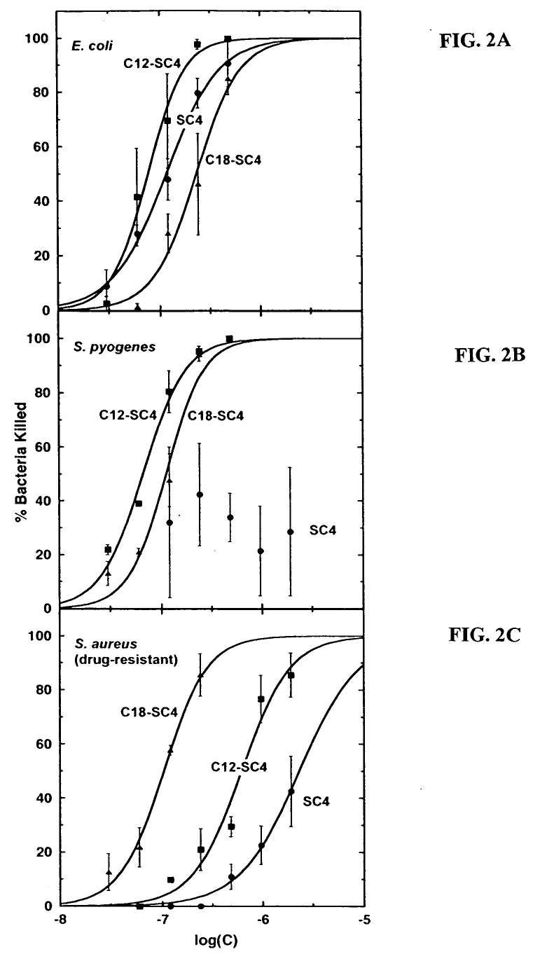

Bactericidal activity. SC4 peptide, and C12-SC4 and C18-SC4 peptide-amphiphiles (FIG. 1) were tested for bactericidal activity against several clinically-relevant strains of Gram-negative, Gram-positive, and drug-resistant bacteria. Activities are reported as the peptide concentration that kills 50% of bacteria (LD₅₀) as determined by sigmoidal fits of dose-response data (exemplified in FIG. 2). Both C12-SC4 and C18-SC4 generally showed increased bactericidal activity relative to SC4 (Table 1). The maximal increase in bactericidal activity as a result of fatty acid conjugation was greater than 30-fold against S. aureus and drug-resistant S. aureus strains. Even though the effective increase against S. pyogenes could not be calculated accurately due to lack of activity from SC4, the increase was greater than 20-fold using the maximum concentration of SC4 tested (2.0 mM). Relative to SC4, C12-SC4 and C18-SC4 showed little, if any, increased activity against Gram-negative strains, with LD₅₀ values varying by a factor of two or less against E. coli and remaining essentially unchanged against P. aeruginosa.

TABLE 1
Biological activities of SC4 peptide and amphiphiles.

| | Bactericidal Activity (LD₅₀, μM)^{a} | | | | | | | | | | | |
|---|---|---|---|---|---|---|---|---|---|---|---|---|
| | E. coli | | P. aeruginosa | S. aureus | | | | S. pyogenes | | | LPS Binding | Hemolysis |
| | J96 | IA2 | Type I | MN8 | MNHO | M49780^{b} | W73134^{b} | Eaton | Wilson | B. anthracis | (IC₅₀, μM) | (LD₅₀, μM) |
| SC4 | 0.12 | 0.21 | 0.028 | 1.3 | 2.3 | 2.0 | 2.3 | >2.0 | >1.0 | >15 | 0.59 | >375 |
| C12-SC4 | 0.077 | 0.079 | 0.035 | 0.68 | 0.34 | 0.66 | 0.62 | 0.066 | 0.16 | 3.25 | 0.23 | 6.2 |
| C18-SC4 | 0.23 | 0.19 | 0.042 | 0.11 | 0.068 | 0.24 | 0.10 | 0.11 | 0.13 | 2.23 | 0.082 | 1.9 |

^{a}all strains are clinical isolates
^{b}strains show resistance to all conventional antibiotics except vancomycin

LPS neutralization. Lysis of Gram-negative bacteria produces the endotoxin lipopolysaccharide (LPS), which, when released in the body in high enough amounts, can trigger the pathologic disorder endotoxemia and lead to sepsis. In this respect, peptides that are both bactericidal and effectively neutralize LPS are of considerable pharmaceutical importance. LPS binding and neutralizing activities of SC4, C12-SC4, and C18-SC4 were measured with the Limulus amebocyte assay. IC₅₀ values were determined from dose response curves. C12-SC4 and C18-SC4 showed LPS binding activities approximately 3-fold and 6-fold higher, respectively, than the SC4 peptide (Table 1). Peptides with higher hydrophobicity tend to bind LPS more strongly, and the hydrophobic nature of the tail groups in SC4 amphiphiles makes binding to the lipid A portion of LPS the most likely site for interaction and subsequent neutralization.

Eukaryotic cell lysis activity. Because SC4 and its amphiphiles likely disintegrate bacterial cell membranes, their ability to lyse eukaryotic cells was assessed. If administered as an antibiotic in animals, the two main eukaryotic cell types that these agents would encounter in blood vessels are erthrocytes (red blood cells) and the endothelial cells lining the vessel walls. At concentrations up to 0.4 mM, neither SC4, C12-SC4, nor C18-SC4 killed endothelial cells in culture. SC4 also demonstrated little hemolytic activity up to 0.4 mM. However, both C12-SC4 and C18-SC4 lysed erythrocytes in the micromolar range (Table 1); C18-SC4 was roughly 3-fold more hemolytic than C12-SC4.

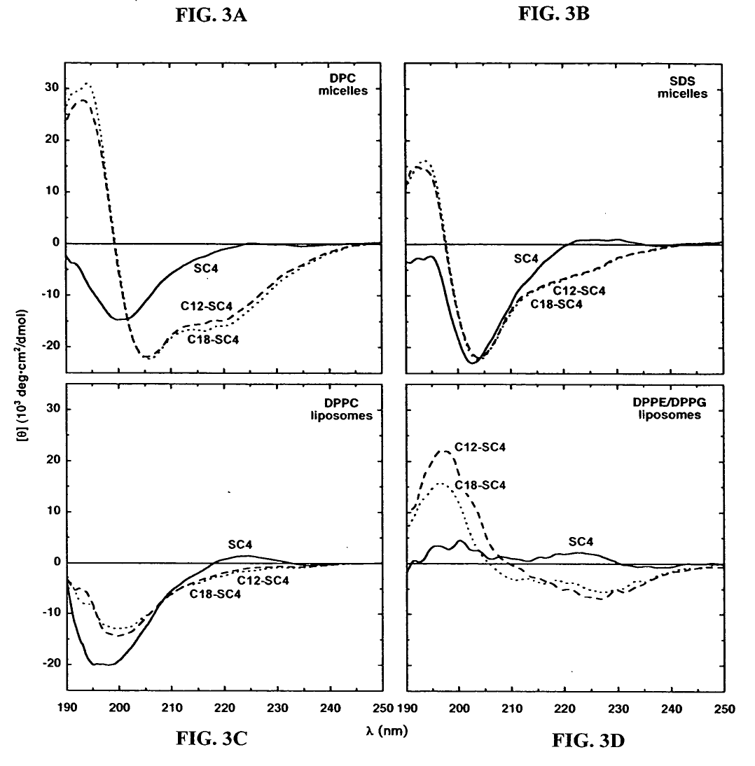

Circular dichroism. For insight into the structural behavior of SC4 peptide-amphiphiles interacting with membranes, the conformation of SC4, C12-SC4, and C18-SC4 in aqueous solution and in the presence of eukaryotic membrane mimics (DPC micelles or DPPC liposomes) and bacterial membrane mimics (SDS micelles or DPPE/DPPG liposomes) was examined by CD spectroscopy (FIG. 3). Aqueous solutions of pure SC4, C12-SC4, and C18-SC4 gave CD spectra consistent with disordered structures; fits of the data using a linear combination of basis spectra indicated 63% to 77% random coil for each of the molecules. Similar CD spectra were observed for SC4 in either DPC or SDS micellar environments, with fits indicating 62-67% coil. However, CD spectra of C12-SC4 and C18-SC4 indicated 78% and 82% ct-helix, respectively, in DPC micelles, and 53% and 54% α-helix, respectively, in SDS micelles.

In eukaryotic membrane-mimicking 1,2-dipalmitoyl-sn-glycero-phosphocholine (PC) liposomes, none of the SC4 molecules showed significant helical structure (76% coil for SC4 and 60% coil for C12- and C18-SC4). In bacterial membrane-mimicking 70% 1,2-dipalmitoyl-sn-glycero-3-phosphoethanolamine/30% 1,2dipalmitoyl-sn-glycero-3-[phospho-rac-(1-glycerol)] (PE/PG) liposomes, the quality of CD spectra was relatively poor due to formation of hazy solutions when SC4 or its amphiphiles were mixed with PE/PG liposome solutions. This effect was minimized, though not eliminated, by using solutions of lower peptide and lipid concentrations. SC4 spectra were particularly noisy and could not be readily interpreted other than to say that above 215 nm the spectrum looked similar to that in PC. CD spectra of C12-SC4 and C18-SC4 were distinct from their corresponding PC spectra. Both C12-SC4 and C18-SC4 in PE/PG gave CD spectra consistent with structured peptides. Qualitatively, the overall shape of these CD traces suggests the presence of significant a-helix conformation.

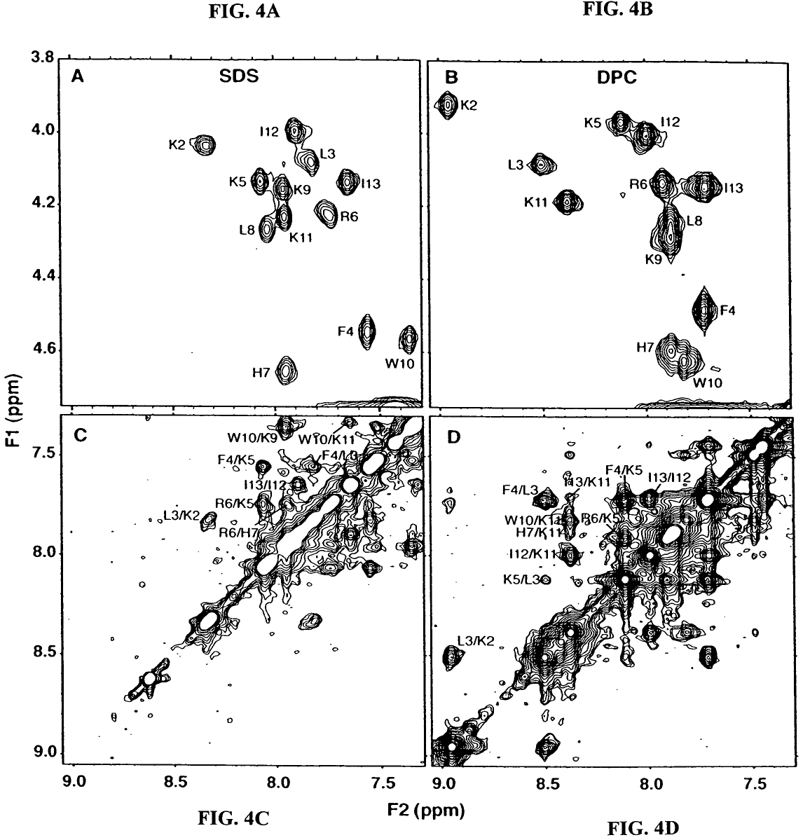

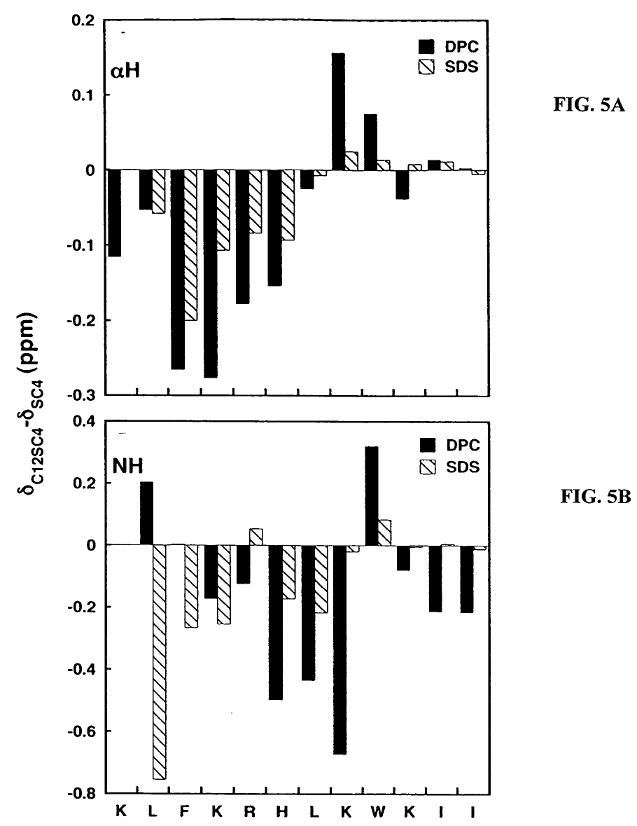

NMR studies. NMR studies were performed on C12-SC4 peptide-amphiphiles in SDS and DPC micellar environments as a result of the overall improved solution behavior and the smaller aggregate size than liposome systems. TOCSY and NOESY spectra of C12-SC4 in DPC and SDS showed well resolved and well dispersed cross-peaks in the H-NH and NH-NH regions (FIG. 4), indicative of the presence of stable peptide conformation. C12-SC4 H and NH resonances are generally shifted upfield relative to SC4 in the same micellar solutions, particularly H resonances belonging to residues K1 through H7 and NH resonances belonging to residues L2 through K8 (FIG. 5). The nature of these shifts suggests that the presence of the acyl chain in C12-SC4, on interacting with these micellar systems, has induced a more stable helix within this N-terminal region of the peptide (Wishart et al., Biochemistry 31, 1647-1651 (1992)). It should be noted, however, that F4 and W9 aromatics may induce ring current shifting of various resonances to attenuate actual upfield shifts of some H or NH resonances; this may help explain why some H and NH shift differences are either zero or positive. H and NH shift differences are generally greater for the peptideamphiphile in DPC than in SDS, which suggests increased helix stability of C12-SC4 in DPC. NOESY data support this conclusion in that long-range NOEs are both more numerous and more intense for C12-SC4 in the presence of DPC micelles than in SDS micelles.

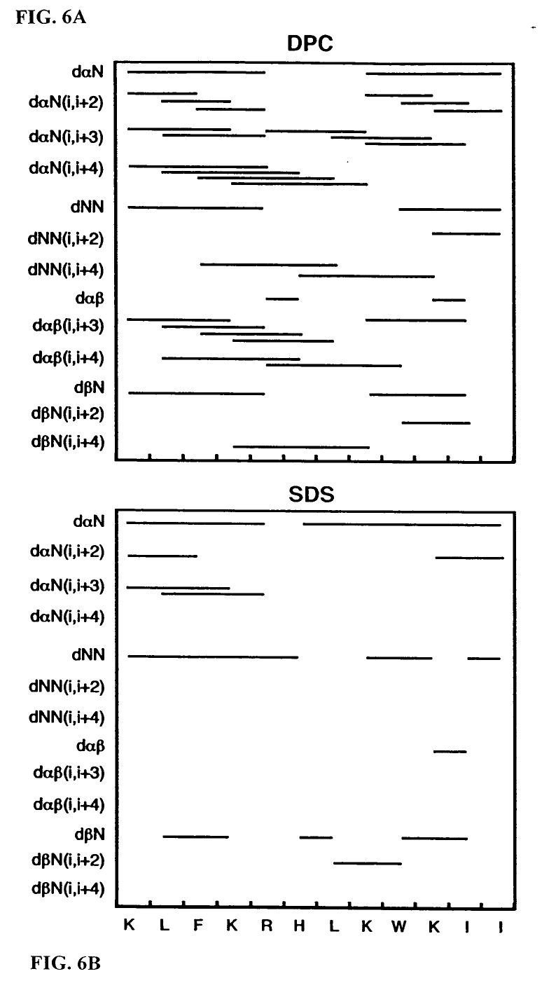

NMR conformational modeling. In order to characterize the conformation of the SC4 amphiphiles in more detail, a full NMR structure analysis was performed on C12-SC4 in DPC micelles at 25° C., conditions that showed the highest level of α-helical content in our CD analysis. The pattern of NOEs observed for C1 2-SC4 was consistent with α-helical conformation (FIG. 6).

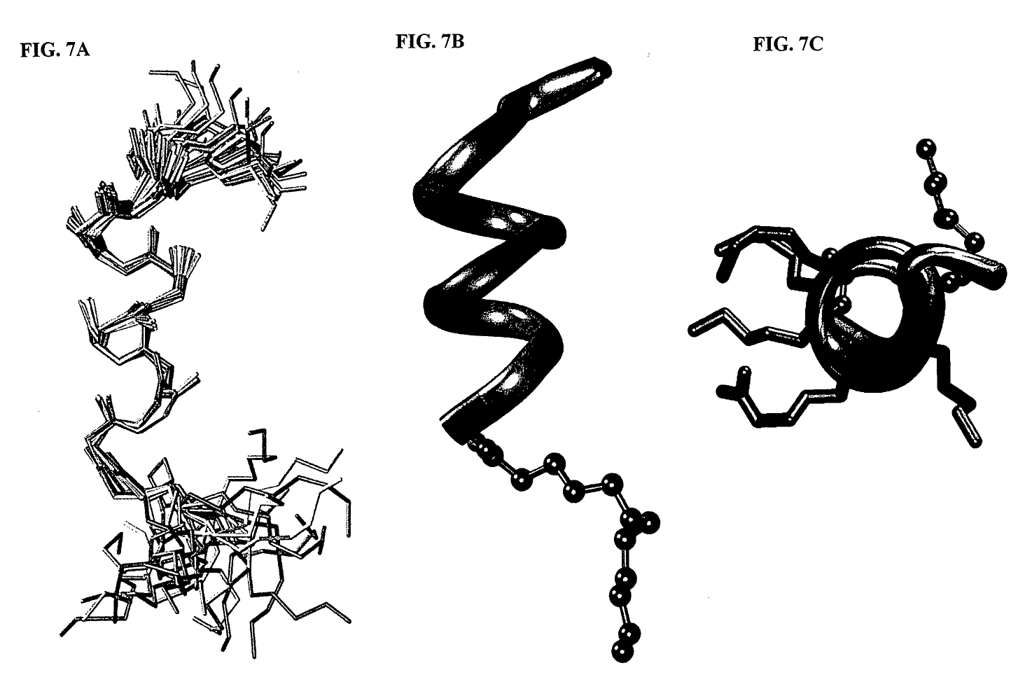

Conformational modeling was performed using NOE distance restraints obtained from NOESY experiments. A total of 196 NOE distance constraints were derived from analysis of NOESY spectra, including 23 intraresidue, 83 sequential, and 90 medium-range (Ii−jI<5) constraints. In addition, 4 hydrogen bonds could be identified by inspection of initial C12-SC4 structures and from long-lived backbone NH resonances, giving rise to 8 hydrogen bond distance constraints. The total number of experimentally derived constraints was therefore 204, an average of 17 constraints per residue. One-hundred structures were calculated using NOE and H-bond constraints; 24 final structures with no NOE violations greater than 0.5 Å were obtained (FIG. 7A). Structural statistics (Table 2) show that the structures satisfy experimental constraints well. Together, the above data indicate that the structures used to represent the solution conformation of C12-SC4 are well converged. The average RMSD for backbone heavy atoms of α-helical residues was 0.24 Å, for all backbone heavy atoms was 0.80 Å, and for the entire molecule (excluding the fatty acid tail) was 1.453 Å. The NMR structure of C12-SC4 in DPC micelles showed an amphipathic, α-helical conformation over much of the length of the peptide (FIG. 7B and FIG. 7C).

TABLE 2
Structural statistics of NOE-derived C12-SC4 structures.^{a}

| | RMS Deviations | Energy (J/mol) |
|---|---|---|
| NOE | 0.1666 ± 0.0039 Å | 117.96 ± 5.53 |
| Angles | 0.9334 ± 0.0218° | 71.56 ± 3.37 |
| Bonds | 0.0085 ± 0.0002 Å | 21.01 ± 1.06 |
| Impropers | 0.7675 ± 0.0619° | 13.54 ± 2.03 |
| Total^{b} | 1.453 Å | 255.40 ± 6.42 |

^{a}None of the 24 final structures exhibited distance restraint violations greater than 0.5 Å. Values shown are mean ± standard deviation.
^{b}Total RMSD was calculated for heavy atoms and does not include the fatty acid tail.

TABLE 3
Tryptophan fluorescence emission maxima in water and
membrane-mimicking environments.^{a}

| | Water | DPPC^{b} | DPPE/DPPG | DPC^{c} | SDS^{c} |
|---|---|---|---|---|---|
| SC4 | 352.7 ± 0.6 | 353.3 ± 1.2 | 342.0 ± 0.0 | 342 | 341 |
| C12-SC4 | 353.0 ± 1.0 | 348.7 ± 2.1 | 338.0 ± 0.0 | 344 | 341 |
| C18-SC4 | 353.3 ± 1.2 | 349.0 ± 1.7 | 339.7 ± 3.2 | 344 | 342 |

^{a}Emission spectra collected 300-450 nm, exitation @ 280 nm, 25° C., C = 5 μM. Values are peak position (nm) ± standard deviation, n = 3
^{b}Peptide-lipid ratios: DPPC, DPPE/DPPG, 1:20; DPC, 1:500; SDS, 1:1000
^{c}n = 1

Membrane interactions via tryptophan fluorescence spectroscopy. Tryptophan fluorescence spectroscopy was used to probe the environment of the W9 residue of the SC4 molecules in water, liposomes, and micelles (Table 3). The emission maximum for SC4, C12-SC4, and C18-SC4 in water was ˜354 nm. A blue-shift from this maximum suggests that, the tryptophan is in a more hydrophobic environment (Lakowicz, J. (1983) Principles of Fluorescence Spectroscopy, Plenum Press, New York). In PC liposomes (eukaryotic membrane mimic), the position of the emission maximum did not change for SC4, but the maxima for C12-SC4 and C18-SC4 were slightly blue-shifted. In PE/PG liposomes (bacterial membrane mimic), the emission maxima showed strong blue-shifts for all three molecules, with the magnitude of the shift being larger for either SC4 amphiphile than the SC4 peptide. Similar results were obtained at 37° C., the temperature at which bactericidal assays were performed. In DPC or SDS micellar environments, the W9 emission peak is blue-shifted and at levels comparable to those observed in DPPE/DPPG liposomes. This suggests that our NMR structural studies in DPC micelles likely reflect the environment of the C12-SC4 amphiphile in bacterial membranes.

Membrane interactions via NMR chemical shifts. While αH and NH chemical shift differences generally reflect α-helix or β-sheet structure and stability (Wishart et al., Biochemistry 31, 1647-1651 (1992)), side-chain chemical shifts can provide insight into the environment with which the folded peptide is interacting. This can be particularly insightful when shift differences other than CH2 groups in longer side-chains are large.

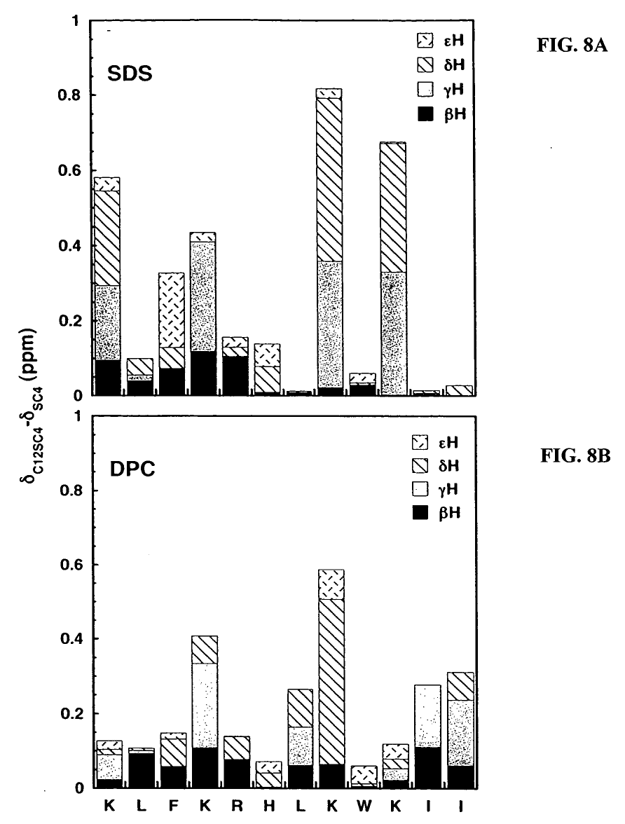

The side-chain chemical shifts were compared for C12-SC4 in micelles (DPC or SDS) with those of the SC4 peptide under the same conditions (FIG. 8); this comparison examines the effect of adding the fatty acid tail to the SC4 peptide. The shift differences associated with γCH and δCH groups are relatively large in the longer side-chain-containing amino acid residues, even compared to their a H, βCH and NH groups (compare to FIG. 5). This indicates that the environment around the side-chains is being significantly perturbed by the presence of the fatty acid tail, likely a combination of conformation change and increased interaction with the detergent micelles. Side-chain chemical shift differences tended to be larger for C12-SC4 in SDS than in DPC. In DPC, the largest chemical shift difference was observed for K5 and K9; shift differences in SDS were also larger for these two residues. The larger shift difference for the C-terminal 111 and 112 residues may indicate a more micelle-buried environment for the terminus of the peptide in DPC. In SDS, large shifts are also found for K1, F3, and K11. The large shift differences of the lysine side-chains in SDS suggest that interactions are primarily between the peptide lysines and the surface negative charges on the SDS micelles. This is also consistent with the smaller shift differences observed for other residues in the peptide.

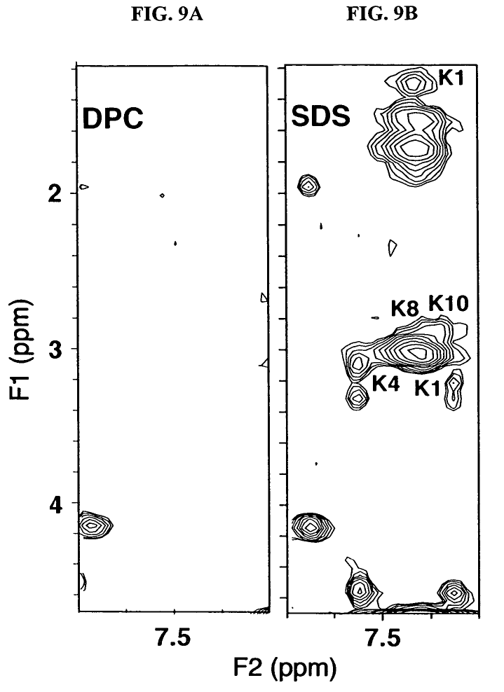

An interaction between lysine side-chains and the SDS micelle surface is also supported by the observation of TOCSY αH, βH, γH, and εH proton cross peaks from terminal side-chain amines of the four C12-SC4 lysine residues in SDS micelles (FIG. 9B). The observation of lysine NH₃⁺ resonances from the peptide in the presence of SDS micelles indicates that κNH protons do not readily exchange with water. Although this could occur if the peptide were buried within an environment of low dielectric, that is, within the micelle, the more likely explanation, especially considering that the crosspeaks are not observed in DPC micelles (FIG. 9A), is that this effect is the result of electrostatic interactions with the micelle surface.

### DISCUSSION

Fatty acid conjugation of the SC4 peptide, itself potently antibacterial, creates an even more potent bactericidal agent and broadens the range of susceptible bacteria to include drug-resistant bacteria, Gram-positive bacteria, and anthrax strains. Conjugation of fatty acids to SC4 increased bactericidal activity most dramatically against Gram-positive bacteria, increasing the activity of SC4 up to 30-fold (FIG. 2 and Table 1). Moreover, drug-resistant Gram-positive strains that are susceptible only to the conventional antibiotic vancomycin were effectively killed at submicromolar concentrations of SC4 peptide-amphiphiles.

The present results indicate the following effect of tail length on bactericidal activity. SC4 conjugated to a C12 fatty acid chain killed Gram-negative bacteria better than SC4 with a C18 chain, whereas SC4 conjugated to a C18 chain typically was more effective against Gram-positive bacteria. The present results suggest that the optimal tail length is, in fact, a function of bacterial species and not a simple rule. This inevitably is related to the composition and architecture of the particular bacterial membrane environment. A simple relationship between tail chain length and increased activity has been similarly elusive in other studies of fatty acid conjugates, although the general conclusions of much of this previous work had suggested an optimal tail length of 11 to 12 carbon atoms (Mak et al., Int. J. Antimicrob. Agents 21, 13-19 (2003); Wakabayashi et al., Antimicrob. Agents Chemother. 43, 1267-1269 (1999); Majerle et al., J. Antimicrob. Chemother. 51, 1159-1165 (2003); Avrahami and Shai, Biochemistry 41, 2254-2263 (2002); and Chicharro et al., Antimicrob. Agents Chemother. 45, 2441-2449 (2001)). SC4 exhibited little, if any, disruptive effects (lysis) on eukaryotic cells up to the millimolar concentration range; however, a relative increase in hemolytic activity in SC4 peptide-amphiphiles was observed (Table 1). This may be due in part, perhaps, to stabilization of an a-helical conformation having a relatively large cationic face. Weiprecht and coworkers observed a similar effect as they increased the angle of the cationic face in model peptides (Wieprecht et al., Biochemistry 36, 12869-12880 (1997)). From a mechanistic standpoint, the increase in hemolytic activity upon addition of the fatty acid tails to SC4 points to a decrease in specificity for bacterial membranes-in other words, a general increase in membrane affinity. This is not surprising given the nonspecific hydrophobic nature of the fatty acid tail. Others have demonstrated similar decreases in specificity as overall peptide hydrophobicity is increased (Wieprecht et al., Biochemistry 36, 6124-6132 (1997)). In contrast to hemolytic activities, no lytic activity of SC4 or its amphiphiles against endothelial cells in culture were observed. It may be that processed red blood cells are simply more susceptible to lytic effects from molecules like peptide-amphiphiles. Nevertheless, for most of the bacterial strains tested, the hemolytic IC₅₀ observed with SC4 peptide-amphiphiles is 10-fold to 100-fold higher than the effective bactericidal LD₅₀, tempering concerns that toxicity might arise when these agents are used in vivo.

The present results suggest that fatty acid conjugation to the peptide increases bactericidal activity by increasing membrane affinity and by enhancing secondary structure in a membrane environment. Peptide-amphiphiles have been shown to stabilize a variety of α-helical and triple helical structures in peptides that are otherwise unstructured (Yu et al., J. Am. Chem. Soc. 120, 9979-9987 (1998); Fields et al., Biopolymers 47, 143-151(1998)), a process that appears to be mediated through self-assembly or incorporation into micelles or liposomes (Gore et al., Langmuir 17, 5352-5360 (2001)). The present CD results demonstrate that SC4 peptide-amphiphiles display similar behavior in the appropriate aggregates (FIG. 3). The SC4 peptide showed CD spectra indicative of random coil conformation under all conditions studied, while both C12-SC4 and C18-SC4 amphiphiles yielded CD spectra consistent with significant helical content in micellar systems and in PE/PG liposomes.

The stabilization of helical secondary structure in SC4 peptide-amphiphiles, may have important functional implications, especially when considering that, at the concentrations examined with the present invention, all SC4 derivatives gave CD traces indicative of random coil, conformation in water. The development of α-helical structure upon interacting with membranes is an important step in the activity of antibacterial peptides (Bechinger et al., Protein Sci. 2, 2077-2084 (1993)), and factors that stabilize helical structure in membrane-bound peptides may therefore assist in developing the amphipathicity apparently required for lysis of the bacterial membrane. On the other hand, peptides that assemble in solution prior to interacting with the membrane have shown reduced antibacterial potency (Houston et al., J. Pept Res 52, 81-88 (1998)). While assembly of SC4 amphiphiles in solution has been observed, such aggregation occurred only well above biologically-relevant concentrations. C12-SC4 and C18-SC4 form aggregates only at concentrations above about 0.5 mM to 5 mM, levels more than 100 times higher than those used in either the biological assays or the biophysical experiments of the present invention. It is possible, however, that the tendency of SC4 amphiphiles to self-aggregate may play a role in permeabilizing cell membranes by allowing membrane-bound amphiphiles to aggregate within the membrane at surface concentrations below that required for the SC4 peptide.

Structural details of the C12-SC4 headgroup are likely as important as conformational stabilization imparted by the presence of the tail. The NMR results in DPC are likely well correlated with the behavior of C12-SC4 in bacterial membranes, based on the similarity of blue shifts observed in tryptophan fluorescence spectra under each condition (Table 3). NMR structural models of C12-SC4 in DPC micelles reveal some of the details of why these molecules are such potent antibacterial agents. The helical secondary structure formed by the peptide shows clear amphipathic character, with a large cationic face and a smaller hydrophobic face on the opposite side of the helix. A large angle subtended by the cationic face of the helix has been shown in model systems to lead to high bactericidal potency relative to peptides with a smaller cationic face (Wieprecht et al., Biochemistry 36, 12869-12880 (1997)). The helix of C12-SC4 is most defined in that portion of the peptide closest to the fatty-acid tail, a result that suggests the increase in helical content observed in CD spectra of SC4 peptide-amphiphiles relative to SC4 is a direct consequence of anchoring of the peptide at the micelle- or liposome-water interface.

The non-specific increase in membrane affinity of C12-SC4 and C18-SC4 is also likely to play a role in bactericidal activity. Recalling that PE/PG liposomes mimic bacterial membranes, and PC liposomes mimic red blood cell membranes, it is easy to draw parallels between our tryptophan fluorescence spectra for SC4 and its amphiphiles and our biological assays with the same molecules. The general increase in SC4 bactericidal activity upon fatty acid conjugation (Table 1) is reflected in the more hydrophobic environment of the tryptophan in C12-SC4 and C18-SC4 in PE/PG and PC liposomes, respectively (Table 3). The enhanced membrane affinity of the SC4 peptide-amphiphiles (as displayed by their fluorescence spectra) would seem to explain, at least in part, the increased biological activity of the amphiphiles.

SC4 and C12-SC4 are known to permeabilize bacterial membranes, so it is logical to look toward models of membrane-permeabilizing peptides for insight into the mechanism of SC4 amphiphiles. The basic models used to describe the membrane-disrupting activity of amphipathic, α-helical antibacterial peptides are one, the barrelstave model in which transmembrane pores form via the aggregation of a small number of peptides spanning the bacterial membrane, two, the carpet model in which a large number of peptides aggregate on and solubilize regions of the bacterial membrane, and three, the toroidal pore model, which shares similarities with the carpet model, but the end state is a series of pores lined with lipids and peptides (reviewed in Oren and Shai, Biopolymers 47, 451-463 (1998)).

The data suggest that the initial event in C12-SC4 and C18-SC4 bactericidal action is electrostatic binding to the membrane surface, a first step that is consistent with each of the three models. Electrostatic interactions were observed incidentally as the formation of precipitates (at millimolar peptide concentrations) or solution haze (at micromolar peptide concentrations) upon adding SC4 peptide or amphiphile to solutions of PE/PG liposomes. Both the chemical shifts of C12-SC4 side-chains (FIG. 8) and the presence of TOCSY connectivity between NH protons (FIG. 9) in SDS micelles support the model of electrostatic interactions between lysine sidechains and the SDS micelle surface.

The barrel-stave model is an unlikely choice if only for simple geometric considerations. SC4, with 12 residues, is likely too short to span the bacterial membrane. Additionally, the charge on SC4 is +5, which modeling suggests is too high for formation of stable barrel-like pores (Zemel et al., Biophys. J. 84, 2242-2255 (2003)). However, other evidence also discounts the barrel-stave model. The electrostatic interactions we observed between 12-SC4 and SDS micelles is long-lasting (samples used for the investigations were equilibrated at least several hours), suggesting that the final state also has a significant electrostatic component. Also, the combination of structural and chemical shift data suggest that the helix is probably lying parallel to the surface of the micelle, with the less polar side of the amphipathic helix facing being more solvent exposed. This type of final state is consistent with the toroidal pore model, in which membrane pores are lined with a mixture of membrane lipids and peptides, with peptides extending not entirely through the membrane thickness, as in the barrel-stave model, but penetrating the lipid membrane only a short distance.

The presence of the fatty-acid tail in SC4 peptide-amphiphiles may provide a means of increasing aggregation at the membrane surface, a 2D analog of the aggregation observed for SC4 in solution. In the context of the toroidal pore model, this increase in aggregation behavior would decrease the surface concentration required for membrane perforation relative to the SC4 peptide. The present bactericidal assays support this model, at least for Gram-positive organisms, as the bactericidal concentration is lower for the SC4 amphiphiles than for the peptide. Another potential influence of the fatty acid tail is insertion into the bacterial membrane with the hydrophobic face of the peptide, perhaps influencing local curvature and inducing toroidal pores. This would also enhance bactericidal activity in the manner observed.

The present invention has shown that fatty acid conjugates of SC4 are potent, broad-spectrum antibacterial agents. The fatty acid tail increases biological activity of the SC4 peptide, both in terms of bactericidal activity and binding to LPS endotoxin, although the most dramatic increases were observed against Gram-positive bacteria. Structural analysis by CD and NMR suggest that fatty acid conjugation increases bactericidal activity by enhancing amphipathic helix formation in membrane-bound SC4 peptide-amphiphiles. Analysis of membrane interactions with NMR and fluorescence suggests that a second effect of fatty acid conjugation is an increase in membrane affinity, which leads to more potent bactericidal activity.

### Example 2

### N-Terminal Acylation Improves Antibacterial Activity

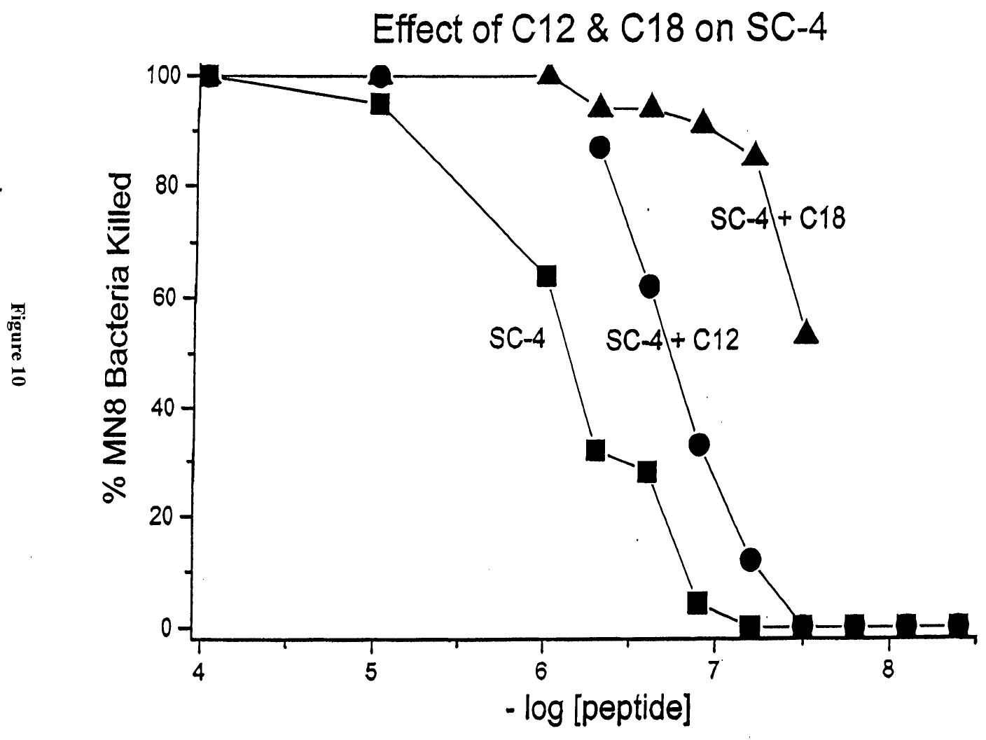

Following procedures outlined in Example 1, the antibacterial effect of the SC-4 peptide and the C12-SC4 and C18-SC4 N-terminal acylation of SC4 on five different Gram-positive bacterial strains was determined. The bacterial strains used were MN8, Hoch, Knutson, FR1722, and RN6390 (Lockwood et al., Biochem. J. 378, 93-103 (2004). Dose response results are shown in FIG. 10 to FIG. 14, respectively.

These results further demonstrate that N-terminal acylation (C12 and C18) of SC4 greatly improves the anti-bacterial activity of the SC-4 peptide. This antibacterial effect tends to be specific for Gram-positive bacteria, including staph and anthrax strains. Activity against Gram-negative strains is increased by only about two-fold at best. This is consistent with known differences in these bacterial membranes through which SC-4 and this entire class of new antibacterial agents works.

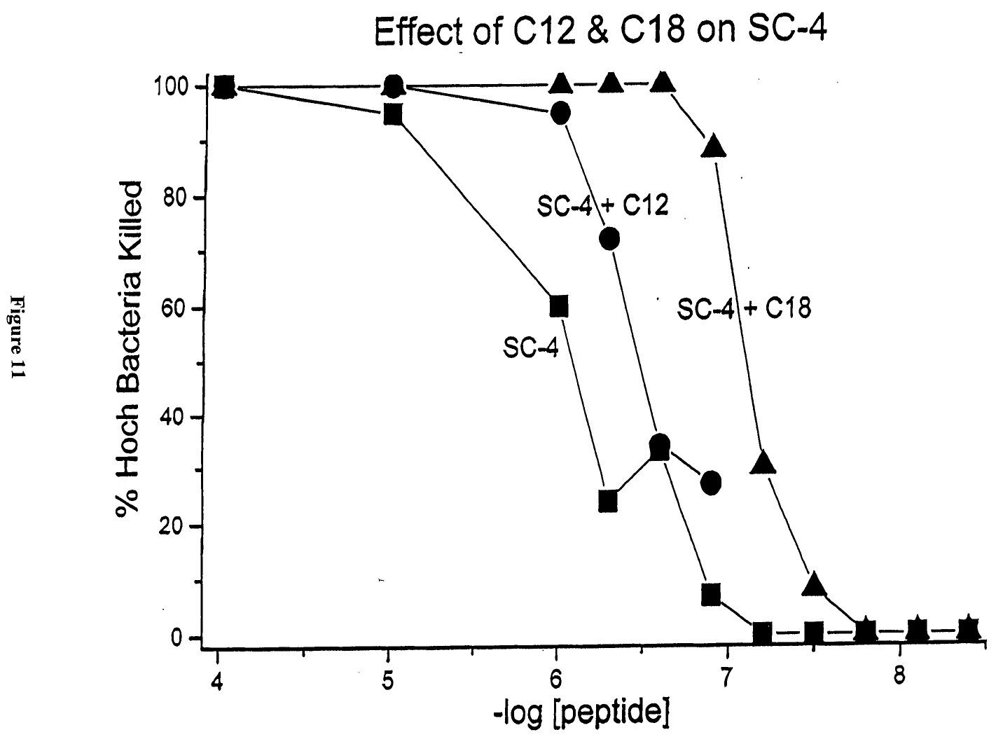

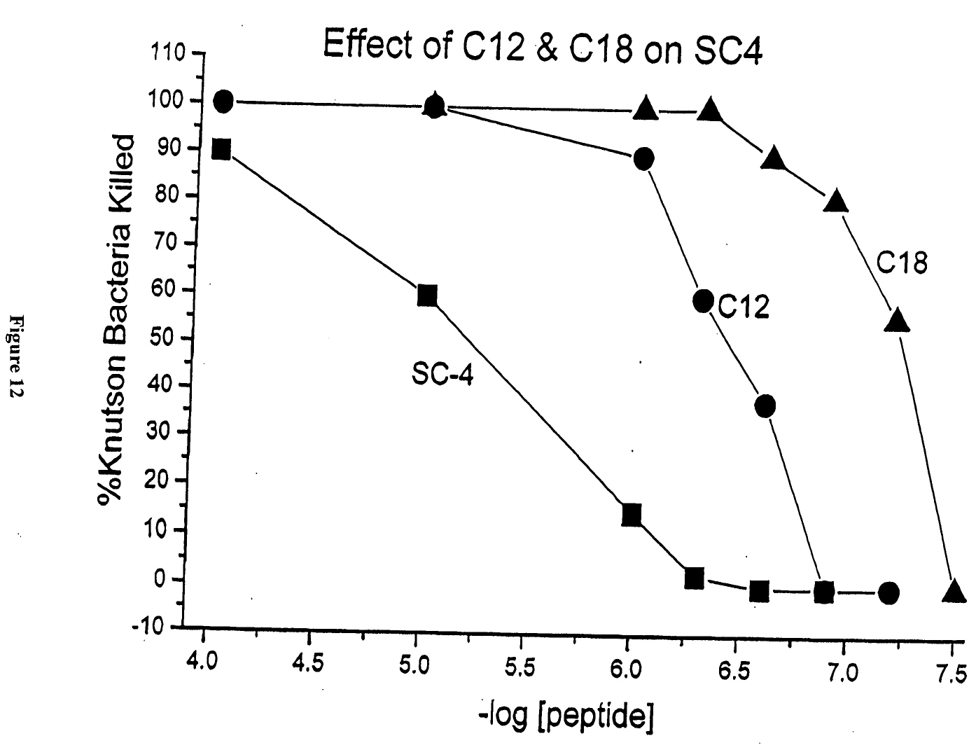

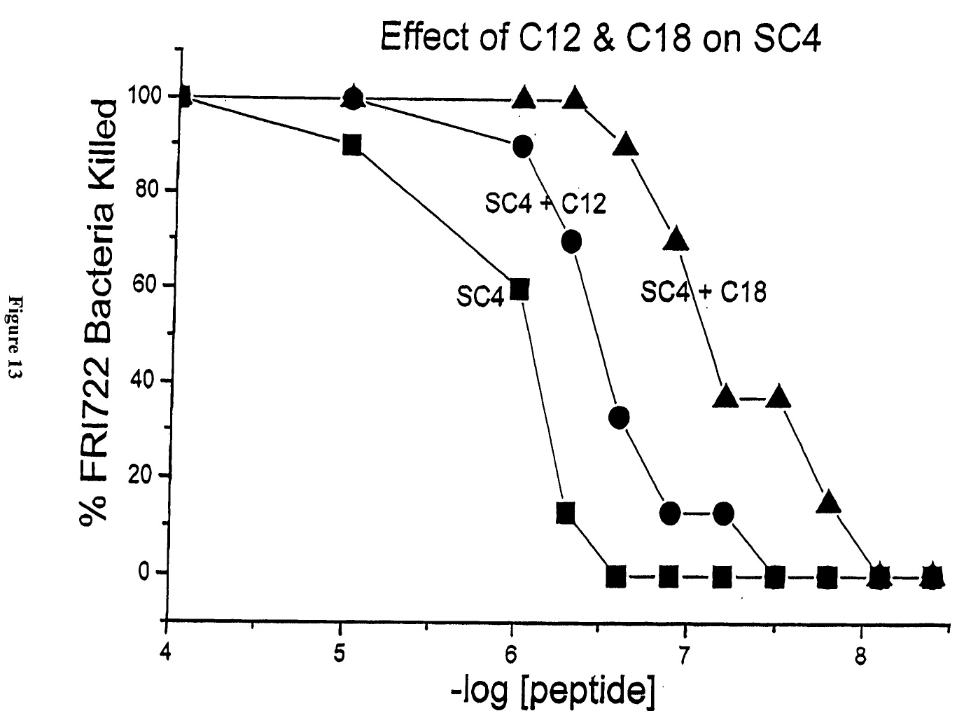

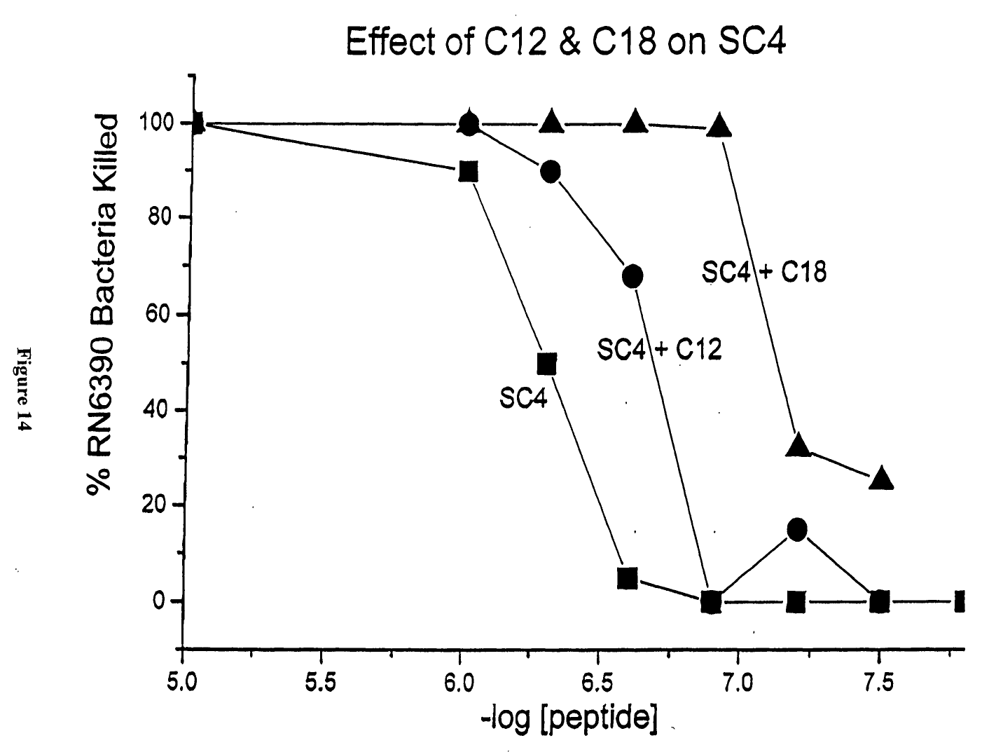

For the C18 derivative, ten to twenty fold increases in antibacterial effect were observed on five different gram-positive bacterial strains; MN8 (FIG. 10), Hoch (FIG. 11), Knutson (FIG. 12), FR1722 (FIG. 13), and RN6390 (FIG. 14).

In addition, SC-4, C12-SC4, and C18-SC4 were tested against two drug-resistant strains of S. aureus (W73134 and M49780, as described in Example 1).

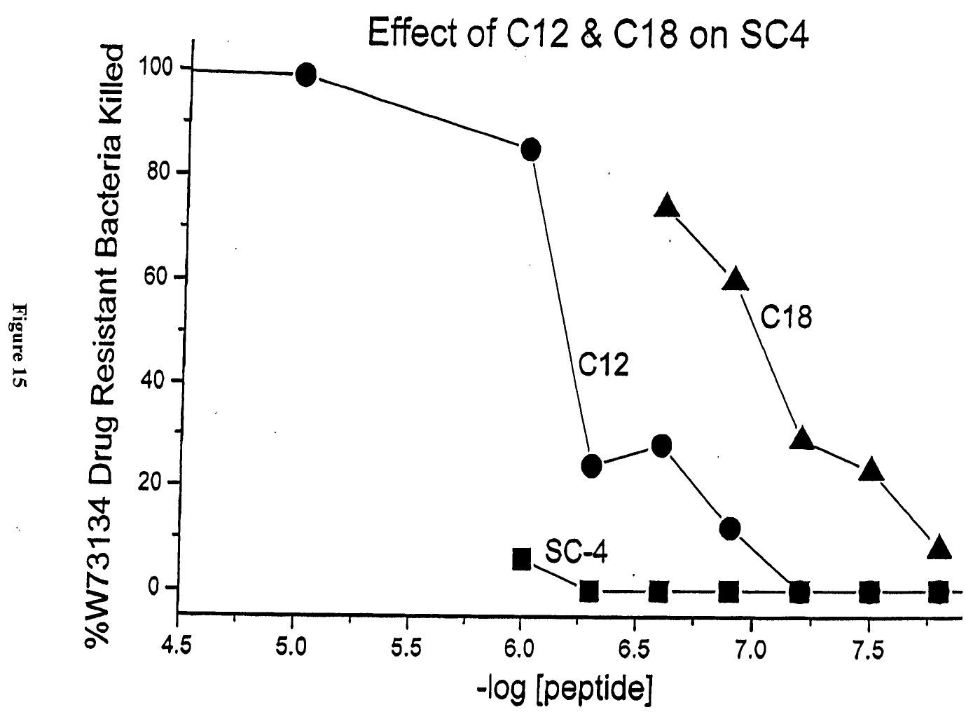

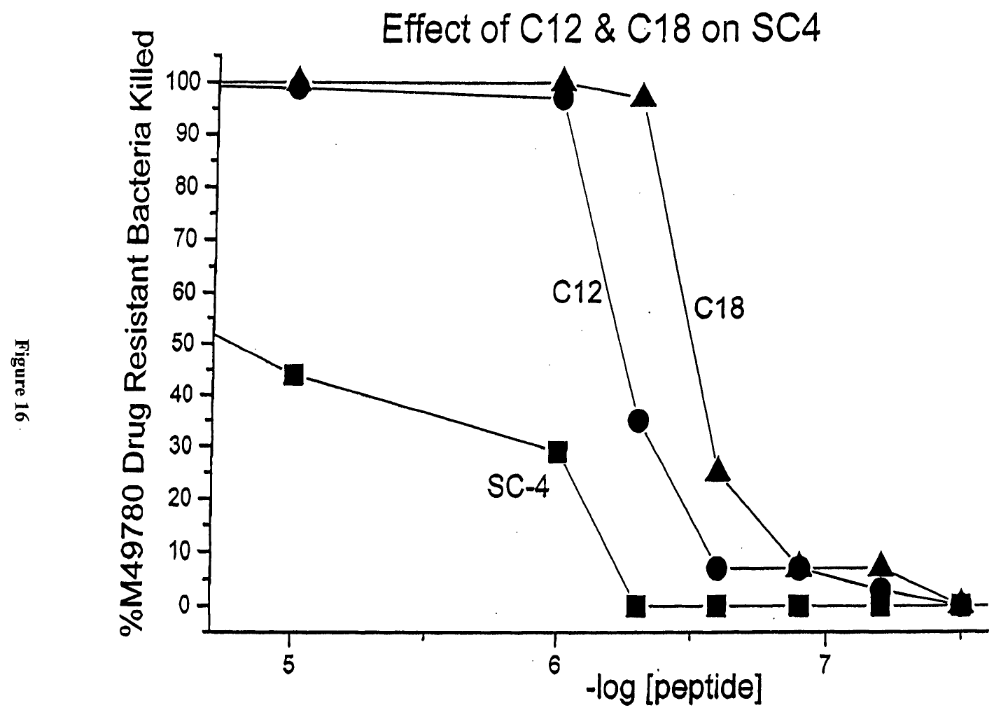

Drug resistance is a big problem with currently available antibiotics. In both cases, C12-and C18 derivatives of SC-4 killed both drug-resistant strains. Both strains are normally only be killed using the most potent commercially available antibiotic, vancomycin. Dose response results for these two Gram-positive drug-resistant strains (FIG. 15 and 16) demonstrate the effects of C12 and C18 modified SC-4, compared to parent SC-4.

### Example 3

### Promotion of Peptide Antimicrobial Activity by Fatty Acid Conjugation

Three peptides, YGAA[KKAAKAA](2) (SEQ ID NO:20), also referred to as “AKK,” KLFKRHLKWKII (SC4), and YG[AKAKAAKA](2) (SEQ ID NO:2 1), also referred to as “KAK,” were conjugated with lauric acid and tested for the effect on their structure, antibacterial activity, and eukaryotic cell toxicity (Chu-Kung et al., Bioconjug Chem. 15(3):530-5 (2004). The conjugated AKK and SC4 peptides showed increased antimicrobial activity relative to unconjugated peptides, but the conjugated KAK peptide did not. The circular dichroism spectrum of AKK showed a significantly larger increase in its alpha-helical content in the conjugated form than peptide KAK in a solution containing phosphatidylethanolamine/phosphotidylglycerol vesicles, which mimics bacterial membranes. The KAK and AKK peptides and their corresponding fatty acid conjugates showed little change in their structure in the presence of phosphatidylcholine vesicles, which mimic the cell membrane of eukaryotic cells. The hemolytic activity of the KAK and AKK peptides and conjugates was low. However, the SC4 fatty acid conjugate showed a large increase in hemolytic activity and a corresponding increase in helical content in the presence of phosphatidylcholine vesicles. These results indicate that antimicrobial peptide hemolytic and antimicrobial activity are associated with changes in secondary structure and improve the ability of the modified peptides to interact with lipid membranes. Fatty acid conjugation will improve the usefulness of peptides as antimicrobial agents by enhancing their ability to form secondary structures upon interacting with the bacterial membranes.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

SEQUENCE FREE TEXT

| | |
|---|---|
| SEQ ID NO: 1-17, 19-21 | Synthetic polypeptides |
| SEQ ID NO: 18 | Synthetic Peptide Substrate |

1. A modified polypeptide comprising a polypeptide having an amphipathic α-helical or 3₁₀ helical structure having one surface comprising primarily positively charged amino acid residues and an opposing surface comprising primarily hydrophobic amino acid residues, wherein these residues define a surface active domain, wherein the polypeptide has up to 14 amino acid residues:
wherein the polypeptide has been modified at the N-terminus and/or C-terminus to include a linear or branched aliphatic group having at least 6 carbon atoms; and
wherein the modified polypeptide demonstrates enhanced bactericidal activity compared to the bactericidal activity of the polypeptide prior to modification at the N-terminus and/or C-terminus to include a linear or branched aliphatic group having at least 6 carbon atoms.

2. The modified polypeptide of claim 1 having up to 12 amino acid residues.

3. The modified polypeptide of claim 1 wherein the polypeptide is selected from the group consisting of SEQ ID NOs: 1-17 and analogs thereof, wherein X is an amino acid,
and wherein an active analog thereof includes the deletion of one or two contiguous or noncontiguous amino acid residues, the addition of one or two contiguous or noncontiguous amino acids, the substitution of one or two amino acids, chemical modification, and/or enzymatic modification.

4. The modified polypeptide of claim 3 wherein X is norleucine.

5. The modified polypeptide of claim 1, wherein the polypeptide is selected from the group consisting of SEQ ID NOs: 1-8 and active analogs thereof,
wherein an active analog thereof includes the deletion of one or two contiguous or noncontiguous amino acid residues, the addition of one or two contiguous or noncontiguous amino acids, the substitution of one or two amino acids, chemical modification, and/or enzymatic modification.

6. The modified polypeptide of claim 1 wherein the aliphatic group includes one or more unsaturated carbon-carbon bonds.

7. The modified polypeptide of claim 1 wherein the aliphatic group is bonded to the polypeptide at the N-terminus and/or C-terminus.

8. The modified polypeptide of claim 1 wherein the aliphatic group is an alkyl group derived from a fatty acid.

9. The modified polypeptide of claim 8 wherein the fatty acid is a C8-C22 fatty acid.

10. The modified polypeptide of claim 8 wherein the fatty acid is a C10-C20 fatty acid.

11. The modified polypeptide of claim 1 wherein the aliphatic group has at least 11 carbon atoms.

12. The modified polypeptide of claim 1 wherein the aliphatic group has 11 to 19 carbon atoms.

13. A modified polypeptide comprising a polypeptide selected from the group consisting of SEQ ID NOs: 1-17 and active analogs thereof, wherein X is an amino acid,
wherein the polypeptide has been modified at the N-terminus and/or C-terminus to include a linear or branched aliphatic group having at least 6 carbon atoms,
wherein an active analog includes the deletion of one or two contiguous or noncontiguous amino acid residues, the addition of one or two contiguous or noncontiguous amino acids, the substitution of one or two amino acids, chemical modification, and/or enzymatic modification.

14. The modified polypeptide of claim 13, wherein the modified polypeptide demonstrates enhanced bactericidal activity compared to the bactericidal activity of the polypeptide prior to modification at the N-terminus and/or C-terminus to include a linear or branched aliphatic group having at least 6 carbon atoms.

15. The modified polypeptide of claim 13, wherein X is norleucine.

16. The modified polypeptide of claim 13, wherein the polypeptide is selected from the group consisting of SEQ ID NOs: 1-8 and active analogs thereof.

17. The modified polypeptide of claim 13, wherein the modified polypeptide is SEQ ID NO:4 or an active analog thereof.

18. The modified polypeptide of claim 13, wherein the modified polypeptide is SEQ ID NO:4.

19. The modified polypeptide of claim 13 wherein the aliphatic group includes one or more unsaturated carbon-carbon bonds.

20. The modified polypeptide of claim 13 wherein the aliphatic group is bonded to the polypeptide at the N-terminus and/or C-terminus.

21. The modified polypeptide of claim 13 wherein the aliphatic group is an alkyl group derived from a fatty acid.

22. The modified polypeptide of claim 21 wherein the fatty acid is a C8-C22 fatty acid.

23. The modified polypeptide of claim 21 wherein the fatty acid is a C10-C20 fatty acid.

24. The modified polypeptide of claim 21 wherein the fatty acid is a C8-C22 fatty acid.

25. The modified polypeptide of claim 13 wherein the aliphatic group has at least 11 carbon atoms.

26. The modified polypeptide of claim 13 wherein the aliphatic group has 11 to 19 carbon atoms.

27. A composition comprising the modified polypeptide of claim 1.

28. A composition comprising the the modified polypeptide of claim 1 and a pharmaceutically acceptable carrier.

29. A modified polypeptide comprising a polypeptide having an amphipathic beta sheet structure having one surface comprising primarily positively charged amino acid residues and an opposing surface comprising primarily hydrophobic amino acid residues, wherein these residues define a surface active domain, wherein the polypeptide has up to 14 amino acid residues;
wherein the polypeptide has been modified at the N-terminus and/or C-terminus to include a linear or branched aliphatic group having at least 6 carbon atoms; and
wherein the modified polypeptide demonstrates enhanced bactericidal activity compared to the bactericidal activity of the polypeptide prior to modification at the N-terminus and/or C-terminus to include a linear or branched aliphatic group having at least 6 carbon atoms.

30. A method for treating a bacterial infection in a subject comprising administering to a subject a modified polypeptide of claim 1 in an amount effective to demonstrate bactericidal activity.

31. The method of claim 30 wherein the modified polypeptide also neutralizes endotoxin.

32. A method for treating endotoxemia in a subject comprising administering to a subject a modified polypeptide of claim 1 in an amount effective to neutralize endotoxin.

33. The method of claim 32 wherein the modified polypeptide also demonstrates bactericidal activity.

34. A method for inhibiting bacterial growth in vitro, the method comprising contacting bacteria with a modified polypeptide of claim 1 in an amount effective to inhibit bacterial cell growth and/or demonstrate bactericidal activity.

35. A method for neutralizing endotoxin in vitro, the method comprising contacting cells with a a modified polypeptide of claim 1 in an amount effective to neutralize endotoxin.

36. A method for decreasing the amount of TNFα in a subject, the method comprising administering to the subject a modified polypeptide of claim 1 in an amount effective to decrease the amount of TNFα.

37. A method for decreasing the amount of TNFα in vitro, the method comprising incubating cells with a modified polypeptide of claim 1 in an amount effective to decrease the amount of TNFα.

38. A method for inhibiting endothelial cell proliferation in a subject, the method comprising administering to the subject a modified polypeptide of claim 1 in an amount effective to inhibit endothelial cell proliferation.

39. A method for inhibiting endothelial cell proliferation in vitro, the method comprising contracting endothelial cells with a modified polypeptide of claim 1 in an amount effective to inhibit endothelial cell proliferation.

40. A method for inhibiting angiogenic-factor mediated inter-cellular adhesion molecule expression down-regulation in a subject, the method comprising administering to the subject a modified polypeptide of claim 1 in an amount effective to inhibit angiogenic-factor mediated inter-cellular adhesion molecule expression down-regulation.

41. A method for inhibiting angiogenic-factor mediated inter-cellular adhesion molecule expression down-regulation in vitro, the method comprising contacting endothelial cells with a modified polypeptide of claim 1 in an amount effective to inhibit angiogenic-factor mediated inter-cellular adhesion molecule expression down-regulation.

42. A method for inhibiting angiogenesis in a subject, the method comprising administering to the subject a modified polypeptide of claim 1 in an amount effective to inhibit angiogenesis.

43. A method for inhibiting angiogenesis in vitro, the method comprising contacting cells with a modified polypeptide of claim 1 in an amount effective to inhibit angiogenesis.

44. A method for inhibiting tumorigenesis in a subject, the method comprising administering to the subject a modified polypeptide of claim 1 in an amount effective to inhibit tumorigenesis.
